(19) United States
(12) Patent Application Publication
(10) Pub. No.: US 2007/0208269 A1
(43) Pub. Date: Sep. 6, 2007

(54) MASK ASSEMBLY, SYSTEM AND METHOD FOR DETERMINING THE OCCURRENCE OF RESPIRATORY EVENTS USING FRONTAL ELECTRODE ARRAY

(76) Inventors: John Robert Mumford, Mississauga, ON (CA); Ronald Leon Kurtz, Oakville, ON (CA); Jianping Wu, Mississauga, ON (CA)

Correspondence Address: BERESKIN AND PARR, 40 KING STREET WEST, BOX 401, TORONTO, ON, M5H 3Y2, CA

(21) Appl. No.: 11/740,717

(22) Filed: Apr. 26, 2007

Related U.S. Application Data
(63) Continuation-in-part of application No. 11/615,584, filed on Dec. 22, 2006.
(63) Continuation-in-part of application No. 11/435,938, filed on May 18, 2006.
Application No. 11/435,938 is a continuation-in-part of application No. 11/131,284, filed on May 18, 2005.
(60) Provisional application No. 60/571,942, filed on May 18, 2004.
(60) Provisional application No. 60/571,890, filed on May 18, 2004.
(60) Provisional application No. 60/571,944, filed on May 18, 2004.

(51) Int. Cl. A61B 5/04; A61B 5/103; A61B 5/00

(52) U.S. Cl. 600/546; 600/595; 600/544; 600/323

(57) ABSTRACT

Embodiments of the invention relate to methods, systems and mask assemblies for use in determining the occurrence of respiratory events using a frontal electrode array. The methods, systems and mask assemblies involve use of means for flow measurement of breathing gas of a person, blood oxygen saturation measurement means and a frontal electrode array for measuring frontal bioelectric signals, each of which is coupled to a processing unit. The processing unit is configured to determine the occurrence of at least one of an apnea event and a hypopnea event based on the measurement signals. Some embodiments involve calculation of an apnea-hypopnea index (AHI) based on the determined apnea and hypopnea events over a period of time.

### CROSS-REFERENCE TO RELATED APPLICATIONS

This application is a continuation-in-part of U.S. patent application Ser. No. 11/615,584, filed Dec. 22, 2006 and a continuation-in-part of U.S. patent application Ser. No. 11/435,938, filed May 18, 2006, which is a continuation-in-part of U.S. patent application Ser. No. 11/131,284, filed May 18, 2005, which claims the benefit of U.S. Provisional Patent Application Ser. No. 60/571,942, filed May 18, 2004, and the benefit of U.S. Provisional Patent Application Ser. No. 60/571,890, filed on May 18, 2004 and the benefit of U.S. Provisional Patent Application Ser. No. 60/571,944 filed on May 18, 2004, the entire contents of all of which are hereby incorporated by reference.

### TECHNICAL FIELD

The described embodiments relate to a method, system and mask assembly having frontal electrodes for the detection of physiological information and the determination of sleep state as part of determining the occurrence of respiratory events.

### BACKGROUND

Obstructive Sleep Apnea (OSA) is a life-threatening condition characterized by frequent episodes in which an individual stops breathing or breathes less efficiently during sleep. OSA is caused by a blockage of the airway typically resulting from the collapse and closure of the soft tissue in the rear of the throat during sleep. With each apnea event, the brain arouses the individual in order for the individual to resume breathing, but consequently sleep is fragmented and of poor quality.

According to the National Institute of Health, OSA currently affects more than twelve million Americans (4% of men and 2% of women), making this disorder as common as adult diabetes. Further, the disrupted and/or poor quality sleep that is associated with OSA may lead to serious health issues including hypertension, heart disease, diabetes, and stroke. Moreover, untreated sleep apnea may be responsible for job impairment and motor vehicle crashes. For example, the Department of Transport in the UK estimates that 20% of road accidents leading to death and serious injury are caused by drowsiness or sleep disorders.

When an individual is diagnosed with OSA, the individual may be prescribed a therapeutic regime involving the use of a Continuous Positive Airway Pressure (CPAP) device. The CPAP device works by delivering a steady flow of air through a soft, pliable mask worn over the individual's nose. The CPAP device essentially pressurizes the throat of the individual thereby preventing the collapse of the soft tissue and keeping the airways open and allowing the individual to breathe uninterrupted during sleep.

The CPAP device is both loud and uncomfortable and has met with various non-compliance issues. Currently the efficacy of treatment is monitored by measuring the usage statistics of the CPAP. Some more sophisticated models (e.g. DeVilbiss AutoAdjust PAP from Sunrise Medical, Longmont Colo.) are able to record breathing patterns to determine the frequency of abnormal breathing events during application of PAP therapy. These devices cannot report on true apnea or hypopnea events because they lack the ability to determine the sleep state of the patient.

It is desired to address or ameliorate one or more of the shortcomings, disadvantages or problems associated with prior systems or devices, or to at least provide a useful alternative thereto.

### SUMMARY

Certain embodiments relate to a system for determining the occurrence of respiratory events, comprising: a plurality of electrodes positionable at frontal locations on a person's forehead to sense bioelectric signals; a flow measurement device configured to measure respiratory flow of the person; a blood oximeter device for measuring a blood oxygen saturation of the person; and a processor/processing unit coupled to the plurality of electrodes, the flow measurement device and the blood oximeter device. The processor is configured to determine a sleep state of the person based on the bioelectric signals, to determine the occurrence of an apnea event based on the sleep state and the respiratory flow and to determine the occurrence of a hypopnea event based on the respiratory flow and at least one of the blood oxygen saturation and the sleep state of the person.

The sleep state may be one of awake and asleep or it may be one of wake, sleep stage one, sleep stage two, deep sleep, REM sleep and movement time.

The plurality of electrodes may be located on a forehead member for positioning on the forehead. The plurality of electrodes may comprise a first electrode located on a projecting portion of the forehead member for positioning adjacent a nasion area of the head, a second electrode located on the forehead member for positioning over a first lateral forehead area and a third electrode located on the forehead member for positioning over a second lateral forehead area opposite the first lateral forehead area. Conductors may be formed on the forehead member for electrically coupling the first, second and third electrodes to an output connector.

A fourth electrode may be located on the forehead member intermediate the second and third electrodes for positioning over a central forehead area. The second and third electrodes may be located on the forehead member for positioning higher on the forehead than Fp1 and Fp2 electrode positions. The fourth electrode may be located on the forehead member for positioning above the first electrode. The second and third electrodes may be located on the forehead member for positioning laterally beyond respective Fp1 and Fp2 electrode positions. The first, second and third electrodes may be positioned in a triangular configuration. The conductors may comprise a printed flexible material. The second and third electrodes may have a separation of 70 to 110 mm. The first and fourth electrodes may have a separation of 35 to 55 mm.

The second and third electrodes may be located on opposed lateral wings of the forehead member. The output connector may be coupled to one of the lateral wings. The forehead member may comprise a flexible limb extending from the one lateral wing and having the output connector coupled to the conductors at an end of the limb.

Each of the first, second and third electrodes may be formed on a substrate on the forehead member. The forehead member may have an adhesive layer disposed on at least a part of an underside of the forehead member adjacent each of the first, second and third electrodes. The adhesive layer may be disposed on substantially the entire underside of the forehead member where the electrodes are not disposed.

The forehead member may be flexible to accommodate varying forehead shapes. The forehead member may comprise a flexible plastic substrate or a woven material.

The first, second and third electrodes may be removably attachable to the forehead member. Each first, second and third electrode may comprise a connection part for electrically and mechanically connecting to a corresponding part on the flexible member. Each first, second and third electrode may have a portion of adhesive material disposed around the respective electrode for affixing the respective electrode to the skin of the forehead.

The processor may be configured to determine the sleep state based on a plurality of rules applied in relation to the bioelectric signals. A signal conditioning unit may be coupled to the processor for receiving detected electrical potentials from the plurality of electrodes, conditioning the electrical potentials to generate the biological signals and providing the biological signals to the processor. The plurality of rules may be empirically derived based on correlation of physiological conditions with particular biological signals or signal patterns. The electrical potentials may correspond to at least one of EEG, EOG and EMG signals. A pre-processing unit may be coupled to the sensing unit for conditioning the detected electrical potentials.

One or more sensors may be coupled to the processor and located away from the head for detecting movement of a body of the patient. The one or more sensors may comprise one or more accelerometers. The one or more sensors may comprise one or more electromyographic sensors.

The blood oximeter device may be located on the forehead member or on a finger or ear of the person.

An air supply interface may be provided for supplying positive airway pressure to an airway of the person. The air supply interface may comprise a nasal interface or a naso-oral interface.

The processor may be configured to determine a total time duration that the person is in a sleep state in which the person is asleep. The processor may be further configured to determine an apnea-hypopnea index based on the total time duration and the number of occurrences of apnea and hypopnea events during the total time duration. The processor may be further configured to determine whether the person has a sleep disorder condition based on the AHI. The processor may be further configured to determine a therapeutic efficacy of the supplied positive airway pressure based on the AHI. The processor may be further configured to determine the occurrence of an arousal event based on the bioelectric signals.

The processing unit may be further configured to determine the occurrence of a respiratory effort related arousal (RERA) event based on the bioelectric signals and the measured respiratory flow. The processing unit may be further configured to determine a respiratory distress index (RDI) based on the number of occurrences of apnea, hypopnea and RERA events during at least part of the time duration. The processing unit may be further configured to monitor the occurrence of respiratory events during a first part of the designated period without supplying positive airway pressure to the airway and to monitor the occurrence of respiratory events during a second part of the designated period while supplying positive airway pressure to the airway. The processing unit may be further configured to end the first part of the designated period and begin the second part of the designated period in response to determining that one or more criteria are satisfied in relation to the occurrence of respiratory events during the first part of the designated period.

The processing unit may be further configured to determine the occurrence of an arousal event based on the bioelectric signals and further configured to determine the occurrence of the hypopnea event based on the respiratory flow, the sleep state of the person and at least one of the arousal event and the blood oxygen saturation.

Further embodiments relate to a method of determining the occurrence of respiratory events, comprising: positioning a plurality of electrodes at frontal locations on a person's forehead; sensing bioelectric signals using the plurality of electrodes; measuring respiratory flow of the person; measuring a blood oxygen saturation of the person; determining a sleep state of the person based on the bioelectric signals; determining occurrence of an apnea event based on the sleep state and the respiratory flow; and determining occurrence of a hypopnea event based on the respiratory flow and at least one of the blood oxygen saturation and the sleep state of the person.

The method may further comprise determining a total time duration that the person is in a sleep state in which the person is asleep. The method may further comprise determining an apnea-hypopnea index based on the total time duration and the number of occurrences of apnea and hypopnea events during the total time duration. The method may further comprise determining whether the person has a sleep disorder condition based on the AHI. The method may further comprise supplying positive airway pressure to an airway of the person. The method may further comprise determining a therapeutic efficacy of the supplied airway pressure based on the AHI.

The method may further comprise determining occurrence of a respiratory effort related arousal (RERA) event based on the bioelectric signals and the measure respiratory flow. The method may further comprise determining a respiratory distress index (RDI) based on the number of occurrences of apnea, hypopnea and RERA events during at least part of the time duration.

The method may further comprise monitoring the occurrence of respiratory events during a first part of the designated period without supplying positive airway pressure to an airway of the person and monitoring the occurrence of respiratory events during a second part of the designated period while supplying positive airway pressure to the airway. The method may further comprise ending the first part of the designated period and beginning the second part of the designated period in response to satisfaction of one or more criteria in relation to at least one of: the occurrence of respiratory events during the first part of the designated period; time spent in one or more sleep states; time spent in a body position; and expiry of the first part of the designated period.

Further embodiments relate to a system for determining the occurrence of respiratory events, comprising: a plurality of electrodes positionable at frontal locations on a person's forehead to sense bioelectric signals; a measurement device for measuring respiratory flow of the person; and a processing unit coupled to the plurality of electrodes and the flow measurement device, wherein the processing unit is configured to determine a sleep state of the person based on the bioelectric signals and to determine the occurrence of an arousal based on the bioelectric signals, and wherein the processing unit is further configured to determine the occurrence of an apnea event based on the sleep state and the respiratory flow and to determine the occurrence of a hypopnea event based on the respiratory flow, the sleep state of the person and the occurrence of an arousal.

Still other embodiments relate to a method of determining the occurrence of respiratory events, comprising: positioning a plurality of electrodes at frontal locations on a person's forehead; sensing bioelectric signals using the plurality of electrodes; measuring respiratory flow of the person; determining a sleep state of the person based on the bioelectric signals; determining occurrence of an arousal based on the bioelectric signals; determining occurrence of an apnea event based on the sleep state and the respiratory flow; and determining occurrence of a hypopnea event based on the respiratory flow, the sleep state and the arousal.

### BRIEF DESCRIPTION OF THE DRAWINGS

For a better understanding of the embodiments and to show more clearly how they may be carried into effect, reference will now be made, by way of example only, to the accompanying drawings, in which:

FIG. 1 is a general block diagram of a CPAP system including a mask assembly with integrated sensors in accordance with an embodiment;

FIG. 2a is a front view of a mask assembly with integrated sensors in accordance with another embodiment;

FIG. 2b is a side view of the mask assembly of FIG. 2a;

FIG. 3 is a front view of mask assembly with integrated sensors according to an alternate embodiment;

FIG. 4 is a front view of a mask assembly with integrated sensors according to another embodiment;

FIG. 5 is a front view of a mask assembly with integrated sensors and a remote processing unit according to another embodiment;

FIG. 6 is a block diagram of the remote processing unit of FIG. 5;

FIG. 7 is a block diagram of the remote processing unit of FIG. 5, employing wireless communication according to another embodiment;

FIG. 8 is a block diagram of the monitoring unit of FIG. 1 in accordance with another embodiment.

FIG. 9 is a front view of a mask assembly according to another embodiment;

FIG. 10 is a front view of another mask assembly according to yet another embodiment;

FIG. 11 is a front view of a sensing unit for use in sleep stage determination;

FIG. 12 is an illustrative side cross-section of the sensing unit of FIG. 11, taken along the line A-A;

FIG. 13A is a representative side view of a human head, showing standard electrode positions and electrode positions according to described embodiments;

FIG. 13B is a representative plan view of a human head corresponding to FIG. 13A;

FIG. 14 is a schematic representation of the relative positions of electrodes on the sensing unit;

FIG. 15 is a block diagram of a system for sleep stage determination;

FIG. 16 is a more detailed block diagram of portions of a system for sleep stage determination corresponding to FIG. 15;

FIG. 17 is a block diagram of an alternative configuration of a system for sleep stage determination;

FIG. 18 is a flow chart of a method of sleep stage evaluation according to some embodiments;

FIG. 19 is a flow chart of a method of sleep stage determination according to some embodiments;

FIG. 20 is a block diagram of a system for use in determining the occurrence of respiratory events; and

FIG. 21 is a flowchart of a method of determining the occurrence of respiratory events according to some embodiments.

### DETAILED DESCRIPTION OF EMBODIMENTS

For simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous features or elements. In this description, reference to terms implying a directional orientation, such as lateral, vertical, below, above or downward, are intended to be viewed as if the sensing unit is positioned on a forehead of a human head, while that head is upright. Accordingly, “vertical” is intended to denote directions from the top of the skull toward the neck, while “lateral” is intended to denote positions or directions to one side of a vertical midline of the head extending along the frontal line of symmetry of the face (i.e. perpendicular to vertical). For example, in this context, the eyes are laterally spaced relative to the vertical midline. Thus, “lateral” as applied to the forehead means extending across the forehead between the eyebrows and the hairline and, depending on the shape of the particular forehead, possibly extending around toward the upper temple area.

Terms used herein that imply direction or orientation, such as those mentioned above, are used for ease of description only and are not intended to be a limitation on the described embodiments when they are not in use on the wearer of the mask assembly and/or frontal electrode array.

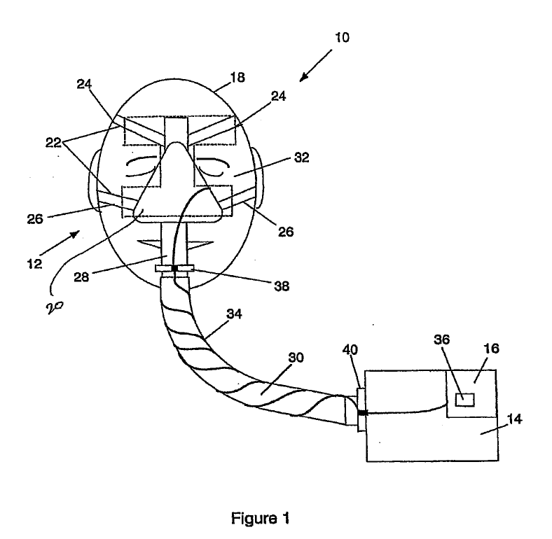

Referring now to FIG. 1, shown therein is a block diagram of a CPAP system 10 including a mask assembly 12 and a CPAP device 14 with an associated monitoring unit 16 for use by a CPAP device wearer 18. The monitoring unit 16 is shown as being an integral part of the CPAP device 14. Other configurations are possible in which the monitoring unit 16 is separate from the CPAP device 14. The mask assembly 12 includes a nasal interface or mask 20 and a harness 22. Harness 22 includes upper straps 24 and lower straps 26. The mask assembly 12 also includes a gas inlet 28 for receiving air, or another suitable gas such as pure oxygen, from the CPAP device 14 via a hose 30. The nasal mask 20 can be made from polystyrene or some other suitable material. The nasal mask 20 may also incorporate a cushion for providing a comfortable and tight fit with the face of the wearer 18.

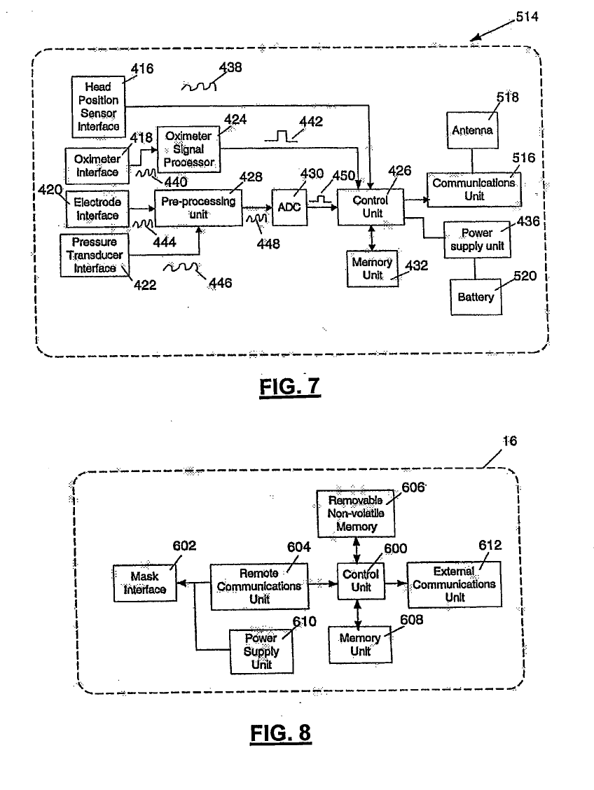

Nasal mask 20 is one exemplary form of nasal interface that can be used with the described embodiments. Other exemplary forms of nasal interface are shown and described in relation to FIGS. 7 and 8. Alternatively, a naso-oral interface, such as is known in the art, may be used in place of the depicted nasal interfaces.

In other embodiments, the harness may include one strap or more than two straps. Further, although the mask assembly 12 is shown having the nasal mask 20, it should be understood that the invention is also applicable to mask assemblies having a nasal/oral mask which covers both the nose and mouth of the CPAP wearer (“user”). Accordingly, the use of the word mask herein refers to both nasal masks and nasal/oral masks.

The mask assembly 12 further includes sensors 32 positioned on the nasal mask 12, the straps 24 and 26 of the harness 22 or on both the nasal mask 12 and the straps 24 and 26. In this exemplary embodiment, the sensors 32 are connected to the monitoring unit 16 via a cable 34. However, the sensors 32 may also be wirelessly coupled to the monitoring unit 16. The sensors 32 include electrodes for detecting one or more of the EEG, EMG or EOG of the CPAP wearer 18. The sensors 32 may further include at least one of a blood oxygen saturation sensor, a body position sensor and a pressure sensor as is described in further detail below.

The physiological information provided by the sensors 32 is pre-processed by the monitoring unit 16 to improve signal quality and then processed according to a sleep efficacy module 36. The sleep efficacy module 36 stores computer program code executable by a processor to monitor the quality of sleep for the wearer 18. This can include determining how long the wearer 18 is in a given sleep state, how many different sleep states the wearer 18 has experienced during sleep, the fragmentation of their sleep states and how many arousals the wearer 18 has experienced. Accordingly, the sleep efficacy module 36 can generate sleep profile information for the wearer 18. The sleep profile information may include data, such as a test score, related to efficacy and compliance. The sleep efficacy module 36 further generates a control signal to control the operational parameters of the CPAP device 14 such as activating or deactivating the CPAP device 14 or altering the amount of pressure that is provided to the gas inlet 28 to improve the quality of sleep of the wearer 18. The sleep efficacy algorithm 36 may use standard techniques, as is commonly known to those skilled in the art, to process the output of some of the sensors (excluding the frontal electrode array described below), determine the quality of sleep and generate the control signal. The sleep efficacy module 36 may identify sleep stages and generate the sleep profile information based on the physiological information sensed from the wearer 18.

The sensors 32 may be integrated directly on the inner surface of the mask assembly 12 rather than being separately attached as is done with conventional CPAP devices. If the sensors 32 are integrated into the mask assembly 12, the sensors 32 do not have to be separately attached by the wearer 18. This ensures that the sensors 32 can be repeatedly applied to the same location on the wearer's face and head every time the wearer 18 wears the mask assembly 12. In addition, the mask assembly 12 can be positioned by the wearer 18, along with the sensors 32, without the aid of a medical professional. Furthermore, since the sensors 32 are already in place, the preparation time prior to going to sleep is reduced for the wearer 18.

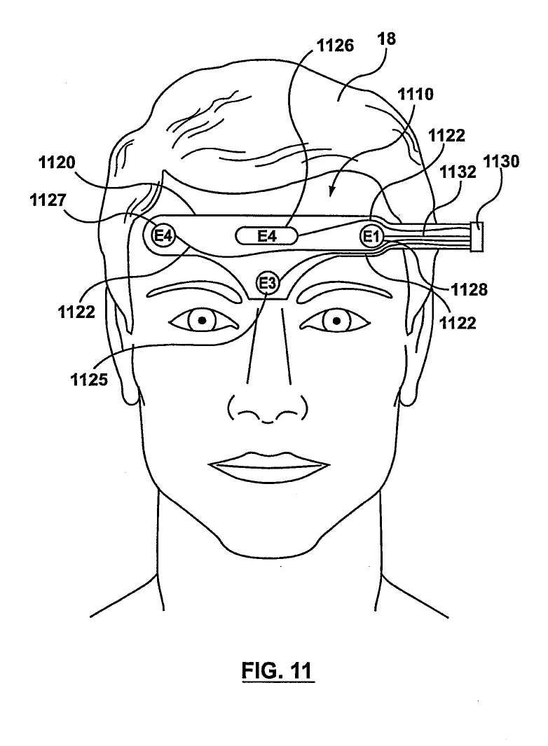

The wiring associated with the sensors 32 may be integrated into a cable 34 that runs along the length of the hose 30. Sensors 32 may have their output coupled to a connector, such as connector 1130 (FIG. 11), for electrically coupling to a corresponding connector on cable 34. The cable 34 may run along the inside or outside of the hose 30. In one embodiment, the cable 34 runs along the inside of the hose 30. In another embodiment, the cable 34 is wound around the outside of the hose 30, as illustrated in FIG. 1. In both instances, the wiring is constricted to the mask assembly 12 instead of hanging loosely on the body of the wearer 18 as is done with conventional CPAP devices. Further, the cable is shielded to reduce the possibility of receiving electromagnetic interference. Connectors 38 and 40 are also provided at either end of the hose 30 so that the hose 30 can be disconnected from the mask assembly 12 and the CPAP device 14 when the mask assembly 12 or the hose 30 requires replacement. This wiring arrangement of the invention provides the wearer 18 with increased mobility and less discomfort. Accordingly, the wearer 18 will enjoy a better quality of sleep. The mask assembly 12 with the integrated sensors 32 is also easy to use and the sensors 32 are automatically engaged when the wearer 18 puts on the mask assembly 12.

In some embodiments, the electrodes that are used as sensors 32 are removably attachable to the mask assembly 12 and configured for placement against the skin of the wearer 18 for sensing the physiological signals. Accordingly, the mask assembly 12 includes attachment means (not shown) for holding the electrodes in place and providing an electrical connection with the cable 34. The implementation of the attachment means depends on the particular type of electrode that is used.

For one type of electrode, the attachment means may be circular apertures, or a cutout portion, with an inner metallic contact, which may be a metallic ring. The apertures are sized to receive cylindrical electrodes which have a plastic portion, a solid conductive gel portion and a metallic conductor disposed there between. The plastic portion is placed in the aperture so that the conductive gel portion is placed against the skin of the wearer 18 when the mask assembly 12 is worn. One example of such an electrode is the Hydrodot™ biosensor, available from Physiometrix Inc. of N. Billerica, Mass., USA. These electrodes require minimal preparation of the skin of the CPAP wearer 18 and the electrodes can be used for several nights before having to be replaced.

Many other types of electrodes may also be used. For instance, metal electrodes can be used which are directly integrated into the mask assembly 12 and do not have to be replaced. In this instance, the wearer 18 may be required to apply a conductive gel to each metallic electrode prior to use. The metallic electrodes may be permanently attached to the mask assembly 12 and would require cleaning each night to remove the old conductive paste before new paste is applied. Saline electrodes may also be used. Saline electrodes have a reservoir that contains saline. Over the course of the night, the reservoir empties. Accordingly, the CPAP wearer 18 must refill the reservoir prior to use of the mask assembly 12. Disk electrodes that are made from gold, silver or carbon may also be used. In addition, peel and stick electrodes that have a layer of silver-silver chloride may also be used. The peel and stick electrodes are likely to need replacement each night. One side of a peel and stick electrode has silver-silver chloride for attachment to the skin, and the other side has a conductive metallic surface. The peel and stick electrode may be held in place by a fastener that ensures that the metallic backing makes electrical contact with a corresponding wire in the mask assembly 12.

The type of electrodes used as some of the sensors 32 does not limit the invention. Regardless of the electrodes used for the sensors 32, it may still be beneficial for the wearer 18 to prepare the skin locations which will receive the electrodes when the wearer 18 wears the mask assembly 12. Accordingly, the wearer 18 may cleanse and slightly abrade their skin with an appropriate cleanser such as NuPrep™ cleanser, available from Weaver & Co. of Aurora Colo., USA. In some instances, the wearer 18 may also apply a conductive paste, such as EC2™ cream for example, to lower the impedance of their skin in order to obtain better physiological signals. EC2™ cream is available from Astro-Med Inc. of West Warwick, R.I., USA. The harness 22 of the mask assembly 12 may be adjusted to apply sufficient pressure to ensure that the electrodes make a good physical contact with the wearer 18.

The electrodes are located at predetermined locations on the face and head of the wearer 18 in order to obtain good signal quality and different types of physiological data with a minimal number of electrodes. Due to the fewer number of electrodes, the mask assembly 12 is easier and more comfortable to wear. The inventors have been able to obtain good physiological data from as little as two electrodes which can provide EEG, EOG or EMG data. This is in contrast to standard sleep staging systems which make use of up to eleven surface electrodes located on the ears, central and occipital lobes, and beside the eyes and on the chin of the wearer.

In some embodiments, the electrodes may be located on the nasion and approximately 4 cm higher on the forehead just above FpZ. The physiological signals obtained from the forehead at these locations provide data related to the CPAP wearer's brainwaves, facial muscle tone and eye movements. Other embodiments use three electrodes, in which one electrode is located at the nasion, another electrode is located just above and to the left of Fp1 and another electrode is located just above and to the right of Fp2. However, other locations, and other combinations of electrodes, may also be suitable as described below.

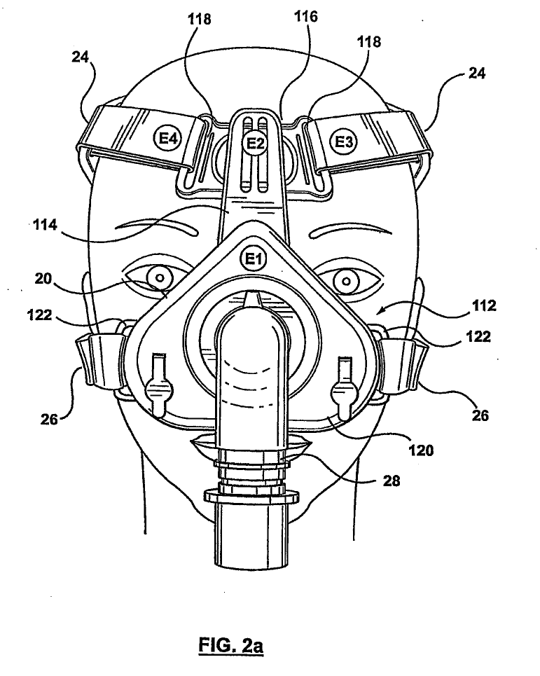

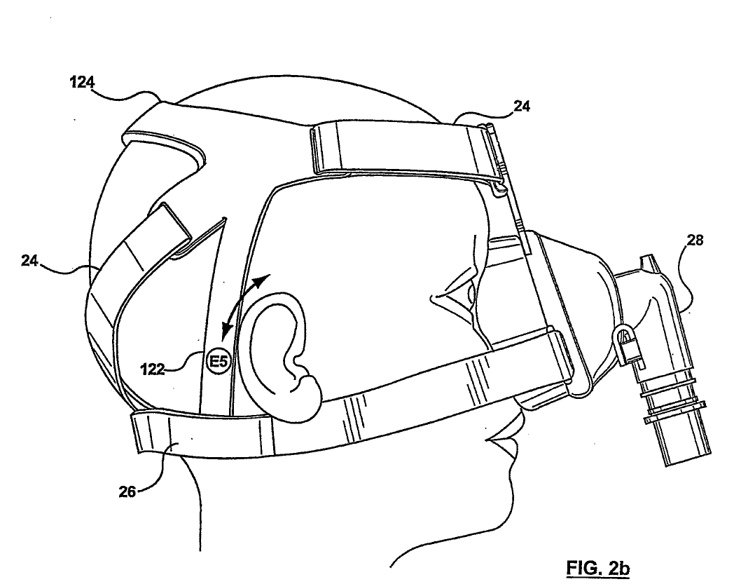

Referring now to FIGS. 2a and 2b, shown therein are front and side views, respectively, of an embodiment of the mask assembly 112 with integrated sensors. The nasal mask 20 of the mask assembly 112 includes a vertical mounting plate 114 that is connected to a forehead support member 116. The forehead support member 116 has two elongated apertures 118 for receiving the straps 24. The mask assembly 112 also includes a flexible seal 120 that rests against the face of the CPAP wearer 18. The seal 120 can be made from an elastomer, urethane foam, rubber or other suitable material and is glued or press fit against the rear of the nasal mask 20.

The harness assembly 22 includes vertical straps 122, on either side of the head of the wearer 18, which connect the upper and lower straps 24 and 26 just behind the ear of the wearer 18. The harness assembly 22 also includes a crown strap 124 that crosses over the crown or vertex of the wearer 18 to connect the upper straps 24 to one another. There are also two elongated apertures 122 disposed at either side near the bottom of the nasal mask 20. The elongated apertures 122 are engaged by the lower straps 26 of the harness assembly 22.

In this embodiment, exemplary locations are shown for an electrode configuration comprising electrodes E1, E2, E3, E4 and E5. Electrodes E1 and E2 are located on the nasal mask 20 and the vertical mounting plate 114 that correspond to the nasion and central forehead regions, respectively, of the CPAP wearer 18. Electrodes E3 and E4 are located on the right and left upper straps 24. Electrode E5 is optional and may be located on the mastoid of the CPAP wearer 18. The electrode E5 may be placed anywhere behind the right or left ear of the wearer 18. Conductive wires are coupled to electrodes E1, E2, E3, E4 and E5 for providing sensed biological signals to CPAP device 14, but are not shown. The separate wires from each electrode E1, E2, E3, E4 and E5 may be bundled together into the cable 34 (or coupled to a connector that connects to cable 34) which runs along the hose 30 of the CPAP system 10 as described above.

The electrode E1 is located at, or approximately 1 cm above, the nasion, which is the depression at the root of the nose of the wearer 18, and is roughly between the eyebrows of the wearer 18. The electrodes E3 and E4 are located below the hairline and spaced apart, laterally positioned between the centerline and the outside of the eyes of the wearer 18. The horizontal and vertical displacements of electrodes E3 and E4 are important for detecting certain EEG information as described below. For instance, if the electrodes E3 and E4 are too close together, then they will not be able to distinguish signals that originate from the deeper structures of the brain. The electrode E5 on the mastoid can help to detect alpha waves in the EEG of the wearer 18 since the electrode E5 is close to the occipital region of the wearer 18. Physiological information from the electrode E5 may be necessary if sufficient information cannot be detected from the frontal electrodes (this depends on the quality of sleep staging performed by the efficacy monitoring module 36).

Electrode locations other than those shown for electrodes E1, E2, E3, E4 and E5 are also possible. For instance it is possible to place one electrode below and beside one eye of the CPAP wearer 18 and the other electrode above and beside the other eye of the CPAP wearer 18. This is the traditional location of EOG electrodes which maximally detect horizontal and vertical eye movements. In addition, it may be possible to vertically flip the location of the electrode E1 with respect to the electrodes E3 and E4. Therefore, rather than forming an inverted triangle pattern, as shown in FIG. 2a, the electrodes E1, E3 and E4 can be oriented in a right side up triangle pattern. This may involve elongating the vertical mounting plate 114.

It is to be noted that each of these electrode locations are on exposed skin surfaces (i.e. not on top of hair) in order to provide a good skin-electrode contact as well as to provide minimal discomfort to the wearer 18. Further, the electrodes are preferably not placed on any large muscles to prevent having the physiological data contaminated with undesirable electromyographic artifacts. Further, the degree to which the locations of the electrodes E1, E2, E3, E4 and E5 can vary depends on the nature of the efficacy monitoring module 36. Small changes on the order of +/−1 cm have little effect. However, it is important to maintain a certain amount of vertical displacement between electrode E1 and the other frontal electrodes E2, E3 and E4. A vertical displacement of as much as 6 cm may be used.

Various subsets of the electrodes may be used in particular embodiments of the invention. One combination may be electrodes E1 and E2. Another combination may be electrodes E1, E3 and E4. Another combination may be electrodes E3, E4 and E5. Another combination may be electrodes E2 and E5. In each of these combinations, there is no reference electrode since one of the electrodes is used to provide both ground and reference signals. This results in a slight reduction in signal quality but the benefit is a reduced number of electrodes. Alternatively, it may be possible to use one of the electrodes as a ground electrode and another of the electrodes as a reference electrode, if necessary. For example, in one combination, electrode E2 may be used to provide a ground signal and electrode E1 may be used to provide the reference signal.

A single channel of physiological information can be derived from two frontal electrodes. However, there is a reduction in the amount of physiological information that is available to determine the sleep stages when only a single channel is used. For instance, with a single channel, detection of eye movements is limited, and EMG information is weak. Also, standard EEG features, such as sawtooth waveforms, spindles, K-complex, alpha and delta waveforms, may be changed. Furthermore, it may be difficult to resolve K-complex signals and spindles from one another using only the electrodes E4 and E3. These signals are more difficult to detect because they do not originate in the frontal lobes of the brain. However, they are useful since they can be used to differentiate between some of the sleep stages. Accordingly, it is preferable, and more robust, although not necessary, to use a subset of electrodes that contains at least three electrodes. However, in some cases it may be possible to use only two electrodes.

The combination of electrodes E1, E3 and E4 provides three channels of physiological data which have a sufficient content of EEG, EMG and EOG information to perform frontal sleep staging (the term “frontal” is used since the physiological data is obtained from the front/face of the wearer 18). One of the three channels is obtained from electrode pair E3 and E1, another of the channels is obtained from electrode pair E4 and E1 and another of the channels is obtained from electrode pair E3 and E4. The data provided by electrode pairs E3 and E1, and E4 and E1 may be used to detect EEG and EOG signals while the data provided by electrode pair E3 and E4 may be used to detect EMG signals. Accordingly, the electrode configuration of electrodes E1, E3 and E4 may be used to detect both horizontal and vertically oriented potentials which is desirable for detecting horizontal and vertical eye movements. Also, dipoles in the brain generate EEG spindles that have different orientations. These EEG spindles, which are helpful for sleep staging, can be detected with electrodes that detect horizontal and vertically oriented potentials.

Two channels are better than a single channel in distinguishing eye blinks from other EEG waveforms, such as K-complex delta activity that is usually less symmetric. With this electrode configuration, eye blinks and rapid eye movements can be used to assist in the detection of wake and REM states since alpha frequencies, which also indicate sleep arousal, originate in the occipital lobe at the rear of the head of the wearer 18 and this is difficult to detect with frontal electrodes. Arousals are also determined by an abrupt increase in alpha and beta band activity of the EEG signals which is evident on the frontal channels. Arousals are important for determining the quality of sleep and the efficacy of therapy.

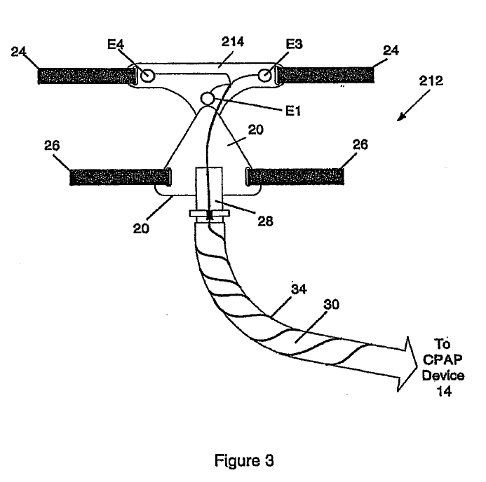

Referring now to FIG. 3, shown therein is a front view of an alternate embodiment of a mask assembly 212 with integrated sensors. In this embodiment, the nasal mask 20 includes a contoured forehead support member 214 with horizontal side wings that extend over the eyebrows of the wearer 18. The electrodes E1, E4 and E3 are all integrated onto the forehead support member 214 of the nasal mask 20 rather than the left and right straps 24. In particular, the electrode E1 is located at the nasion of the wearer 18, the electrode E3 is located near the right horizontal end of the forehead support member 214 horizontally offset with respect to the center of the right eye of the wearer 18, and the electrode E4 is located near the left horizontal end of the forehead support member 214 horizontally offset with respect to the center of the left eye of the wearer 18. Electrodes E3 and E4 are positioned below, but somewhat close to, the hairline of the wearer 18.

Apart from the differences described above, the features, functions and components of mask assembly 212 are otherwise the same as, or similar to, mask assembly 12. Mask assembly 212 may be used within CPAP system 10 in a similar manner to that described above in relation to mask assembly 12.

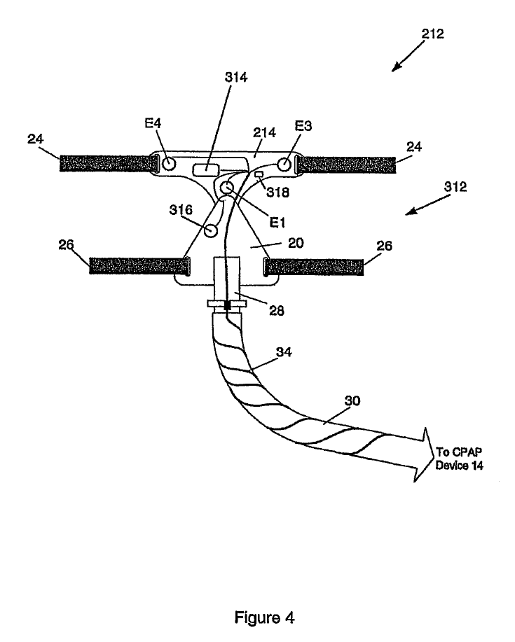

Referring now to FIG. 4, shown therein is a front view of another alternate embodiment of a mask assembly 312 with additional integrated sensors. The mask assembly 312 includes an oximeter sensor 314, a pressure transducer 316 and a position sensor 318. Mask assembly 312 is otherwise the same as mask assembly 212. Not all three additional sensors 314, 316 and 318 may be needed. Additional embodiments are possible in which various subsets of these additional sensors are integrated into the mask assembly 312.

The oximeter sensor 314 provides its output to CPAP device 14 and may be located on the forehead support member 214 in close proximity with the forehead of the wearer 18 to measure the blood oxygen saturation of the wearer 18. Alternatively, the oximeter sensor 314 may be located on an ear clip or inserted into the ear canal and a wire run from the oximeter sensor 314 along one of the straps 24 or 26 and along the nasal mask 20 at which point the wire is integrated within the cable 34.

The pressure transducer 316 is disposed within the nasal mask 20 in close proximity to the gas inlet 28. The position sensor 318 is also preferably located on the forehead support member 214. However, the position sensor 318 may be located within the nasal mask 20; no contact with the skin is required and so the location of the position sensor 318 may be whatever is best suits the ergonomics and manufacturability of the mask assembly 12.

The oximeter sensor 314 may be used to help detect sleep apnea since it provides physiological information from which desaturation and resaturation events in oxygen saturation of the arterial blood of the wearer 18 can be identified. During sleep apnea, there is no air movement into the chest of the wearer 18 and the wearer 18 becomes progressively more hypoxic and hypercarbic. Consequently, respiratory events indicative of OSA may be detected by looking at the rate of change of oxygen desaturations measured during sleep. The oximeter sensor 314 includes light emitting diodes that emit near infrared light at the forehead skin of the wearer 18. The light gets scattered and a portion of the light is reflected to the oximeter sensor 314. The amount of light that gets reflected is related to the spectral absorption of the underlying tissue from which the average oxygenation of the tissue can be derived. Conventional forehead reflectance oximeters may be used, such as the one by Masimo of Irving, Calif., USA to measure peripheral blood oxygenation. Also, the INVOS™ cerebral oximeter made by Somanetics of Troy, Mich., USA may be used as the oximeter sensor 314 to measure oxygenation of the brain.

The pressure transducer 316 can be used to detect the pressure within the cavity of the nasal mask 20 from which the breathing rate (respiration) of the wearer 18 can be derived. The breathing rate of the wearer 18 can be used with other physiological measurements to provide an indication of apnea and hypopnea events. Any suitable pressure transducer with an appropriate size may be used.

The position sensor 318 can be used to detect the position of the head of the CPAP wearer 18. This can be important because the occlusion that occurs during sleep apnea happens mainly when the wearer 18 is lying on his or her back and the soft tissue in the back of the throat collapsing due to gravity. In addition, when the wearer 18 is in the supine position, more effort is required to breathe and consequently additional pressure from the CPAP device 14 is needed. The position of the head relates closely to that of the throat. Accordingly, locating the position sensor 318 on the mask assembly 12 is advantageous. In an alternative, it may be possible to locate a position sensor on the chest of the wearer 18 and run the corresponding wire up to the mask assembly 12 where it is integrated into the cable 34.

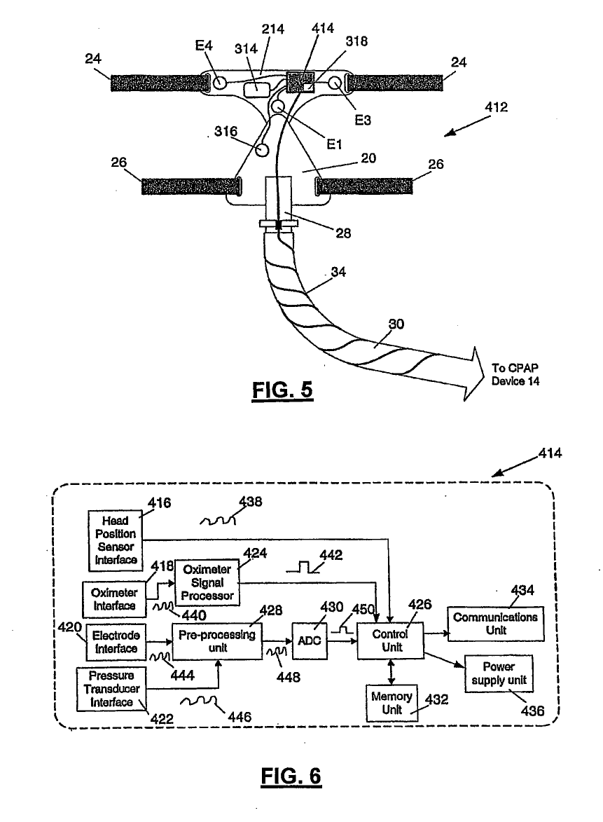

Referring now to FIG. 5, shown therein is a front view of another embodiment of a mask assembly 412 with integrated sensors and a remote processing unit 414 in accordance with the invention. The electrodes E1, E3 and E4, the oximeter sensor 314, the pressure transducer 316 and the position sensor 318 are connected to the remote processing unit 414 which processes the signals provided by these sensors prior to transmitting the signals to the monitoring unit 16 via the cable 34. This results in better quality signals with reduced noise and less contamination caused by motion and electromagnetic interference. The cable 34 may include a power supply connection to provide power to the remote processing unit 414. Alternatively, the remote processing unit 414 may be battery powered. It should be understood that for this embodiment there can be various combinations of the sensors since the oximeter sensor 314, the pressure transducer 316 and the position sensor 318 are optional.

Referring also to FIG. 6, shown therein is a block diagram of the remote processing unit 414. The remote processing unit 414 includes several interfaces for providing an electrical connection with the integrated sensors on the mask assembly 412. The remote processing unit 414 includes a head position sensor interface 416, an oximeter interface 418, an electrode interface 420 and a pressure transducer interface 422 for electrical interface with the appropriate sensor. As mentioned previously, some of the sensors are optional. Accordingly, the remote processing unit 414 may not require each of the interfaces shown in FIG. 6.

The remote processing unit 414 further includes an oximeter signal processor 424 that is connected to the oximeter interface 418 and a control unit 426. The control unit 426 directs the activity of the remote processing unit 414 and processes each of the signals provided by the sensors. The control unit 426 may be a digital signal processor. It should be noted that the oximeter signal processor 424 is optional and the processing performed by the oximeter signal processor 424 may be done by the control unit 426.

The remote processing unit 414 further includes a pre-processing unit 428 that is connected to the electrode interface 420 and an analog-to-digital converter (ADC) 430 that is connected between the pre-processing unit 428 and the control unit 426. The EEG, EMG and EOG signals are very small amplitude signals (in the order of micro-volts) and pre-processing is required to remove noise and amplify these signals. Accordingly, the pre-processing block 428 includes a high-pass filter stage with a cutoff frequency of 0.1 to 1 Hz for removing large DC contact potentials and an amplification stage with a gain in the order of 1,000 V/V for amplifying the electrode signals.

The remote processing unit 414 further includes a memory unit 432 connected to the control unit 426 for storing the measured signals. The memory unit 432 may also be used for storing operational parameters for the remote processing unit 414 as well as programs that are used to process the measured signals. The memory unit 432 is non-volatile and can be a flash memory unit, and the like.

The remote processing unit 414 also includes a host communications unit 434 and a power supply unit 436 connected to the control unit 426. The communications unit 434 directs communication between the remote processing unit 414 and the monitoring unit 16. The communications unit 434 may be a high speed, synchronous serial port such as a UART and the like. The power supply unit 434 is connected to the power wire provided by the cable 34 and processes the power supply signal for use by the remote processing unit 414. The processed power supply signal is provided to the control unit 426 to power the control unit 426 and for distribution to the remaining components of the remote processing unit 426.

It should be noted that the remote processing unit 414 is optional and that all of the signal processing that is done by the remote processing unit 414 may be done by the monitoring unit 16. In this case, the monitoring unit 16 has similar components as those shown in FIG. 6.

In use, the head position interface 416 receives a position signal 438 that is provided by the position interface sensor 318 (position sensors based on mercury switches provide digital signals). The oximeter interface 418 receives an analog oximetry signal 440 from the oximeter sensor 314. The oximeter signal processor 424 processes the analog oximeter signal 440 and provides a processed oximetry signal 442. The electrode interface 420 receives analog electrode signals 444 from the electrodes E1, E3 and E4 and the pressure transducer interface 450 receives an analog pressure signal 446. Both of these analog signals are sent to the pre-processing unit 428 which generates pre-processed signals 448. The pre-processed signals 448 are then digitized by the ADC 430 resulting in digital pre-processed signals 450. The position signal 438, processed oximetry signal 442 and digital pre-processed signals 450 are then sent to the control unit 426.

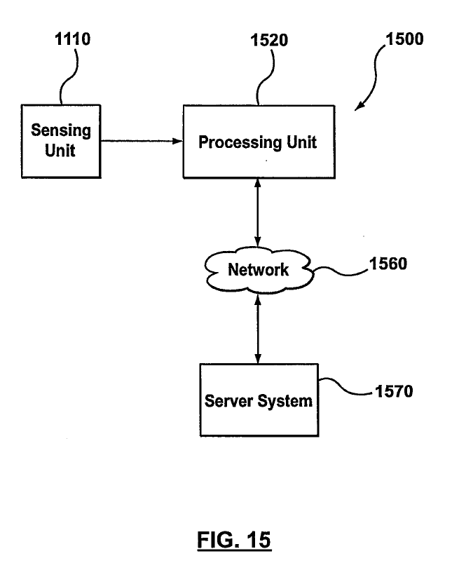

In alternative embodiments, the remote processing unit 414 may also comprise the sleep efficacy module 36 which can be stored in a memory unit 432. Accordingly, the remote processing unit 414 may determine the sleep profile information for the wearer 18, generate the control signal to improve the sleep quality experienced by the wearer 18 and send the control signal to the CPAP device 14 to adjust the pressure that is delivered to the nasal mask 20. In addition, the sleep profile information may be transmitted to a caregiver through a wired connection to a computer. Wireless transmission may also be used as discussed below. The sleep efficacy module 36 employs frontal electrode-based sleep staging to determine the sleep stages of the CPAP wearer 18, as described below in relation to FIGS. 15 to 19.

Referring now to FIG. 7, shown therein is a block diagram of an alternate embodiment of a remote processing unit 514 incorporating a wireless communications unit 516 and an antenna 518 in accordance with the invention. The wireless communication unit 516 runs a suitable wireless personal area network (WPAN) protocol such as the BLUETOOTH™ protocol which is suitable for short-range (i.e. up to 10 meters) communication. For longer-range communication, the wireless communication unit 516 may employ a wireless local area network (WLAN) or wireless wide area network (WWAN) communications protocol. The remote processing unit 514 also includes a battery 520 that is connected to the power supply unit 436. Accordingly, in this case, there is no need for the cable 34.

For the remote processing units 412 and 512, noise is dealt with by selecting amplifiers with a high common mode rejection ratio (CMRR), by having low capacitance isolation of the power supply unit 436 and having a low impedance connection from the electrodes to the skin of the wearer 18. It should be understood that the embodiments for remote processing unit 412 and 512 are exemplary and that some of the components may be combined. For instance, the memory unit 432 and the communications unit 434 may be integrated into the control unit 426.

Referring now to FIG. 8, shown therein is a block diagram of an exemplary embodiment of the monitoring unit 16 of FIG. 1. The monitoring unit 16 may be directly integrated within the CPAP device 14 or it may be separate and work alongside the CPAP device 14. The monitoring unit 16 includes a control unit 600, a mask interface 602, a remote communications unit 604, a removable non-volatile memory 606, a memory unit 608, a power supply unit 610 and an external communications unit 612 connected as shown in FIG. 8.

The control unit 600 controls the operation of the monitoring unit 16 and may comprise one or more of a microprocessor, a digital signal processor, a controller or the like. The mask interface 602 is an interface between the monitoring unit 16 and the sensors on the mask assembly. Accordingly, the mask interface 602 may be an electrical interface with appropriate terminals for receiving the cable 34. Alternatively, in the instances in which the mask assembly includes a wireless remote processing unit, the mask interface 602 may be an antenna. The remote communications unit 604 directs the transmission of data between the mask assembly and the monitoring unit 16. In the instance in which the mask assembly includes a wireless remote processing unit, the remote communications unit 604 employs an appropriate communications protocol such as the BLUETOOTH™ protocol. In the case of a wired connection to the mask, the remote communications unit 604 may be a high speed, synchronous serial port such as a UART and the like.

The control unit 600 receives the data transmitted from the mask assembly. In one embodiment, the control unit 600 may execute the functions of the sleep efficacy module 36, which is stored in the memory unit 608, and generate a control signal for the CPAP device 14. In another embodiment, the remote processing unit may perform the functions of the sleep efficacy module 36, generate the control signal and send the control signal, as well as the sleep profile information, to the control unit 600. In both cases, the control unit 600 sends the control signal to the CPAP device 14 via the external communications unit 612. The external communications unit 612 may also be used to connect to an external computer or network for transfer of the sleep profile information. Accordingly, besides having a connection to the CPAP device 14, the external communications unit 612 may include an Ethernet device, a USB device, a telephone or wireless transceiver or the like for connection to an external computing device or network.

The removable non-volatile memory 606 may store the sleep profile information that includes data, such as test scores, related to the compliance and efficacy of CPAP therapy on the wearer 18. The removable non-volatile memory 606 may also store raw data obtained from the sensors on the mask assembly for inspection by a qualified health professional. The removable non-volatile memory 606 is optional and all of this data may be stored on the memory unit 608.

As mentioned previously, the sleep efficacy module 36 may use a frontal staging algorithm, based on at least the electrode signals, to determine which stage of sleep the wearer 18 is in. The information provided by the electrodes is important because some sleep apnea events occur more frequently in some of the sleep stages rather than others. The sleep stages include sleep stages 1, 2, 3 and 4 and REM sleep. In some individuals, sleep apnea may be more prevalent in the REM stage.

In stage 1 sleep, the EEG is characterized by low voltage, mixed frequency activity, without rapid eye movement and usually with relatively high EMG activity. Stage 2 sleep is characterized by sleep spindles, which are bursts of distinctive waves of 12 to 14 Hz predominantly seen in the central vertex region, as well as K complexes which are delineated, negative, sharp waves immediately followed by positive components lasting more than 0.5 seconds. The K complexes predominantly appear in the central vertex region. REM sleep is characterized by low voltage, mixed frequency EEG activity with the lowest EMG activity and sawtooth waves that appear in the frontal regions of the brain usually in conjunction with bursts of rapid eye movements. Muscle atonia occurs during REM sleep which can affect airway patency and result in increased sleep apnea.

Sleep onset can be determined by the alpha EEG waveform as well as eye blinks (i.e. the lack thereof. Sleep stages 3 and 4 are known as deep sleep states. They are characterized by the dominance of high amplitude (for example, greater than 75 μV) and low frequency (for example, 0.5 to 2 Hz) slow delta activities. Delta activities are predominantly seen in the frontal region.

In some embodiments, the sleep efficacy module 36 activates the CPAP device 14 only once the CPAP wearer 18 falls asleep, thereby easing the transition from wake to sleep, making the therapy more comfortable and improving compliance. The sleep efficacy module 36 may also use the sleep profile information to vary the CPAP titration pressure depending on the sleep stage. For example, more pressure may be delivered in the REM sleep stage in which the incidence of sleep apnea increases due to the relaxation of the throat muscles.

When combined with an automated sleep efficacy module that performs sleep staging and pressure control, the mask assembly of the described embodiments provides a quick, convenient means for monitoring and improving the sleep (quality) profile of the wearer 18. The sleep profile information can be used by physicians to improve the quality of care and allow them to objectively assess the efficacy of treatment and monitor changes to therapy. The efficacy of therapy can be used by employers or law enforcement personnel to prevent hazardous equipment such as cars, airplanes and industrial machines from being operated by individuals who are impaired due to inadequate sleep. The efficacy of therapy can also be used by insurers to determine the need for continued treatment in order to save costs. Further, the sleep profile information can be used to control CPAP therapy.

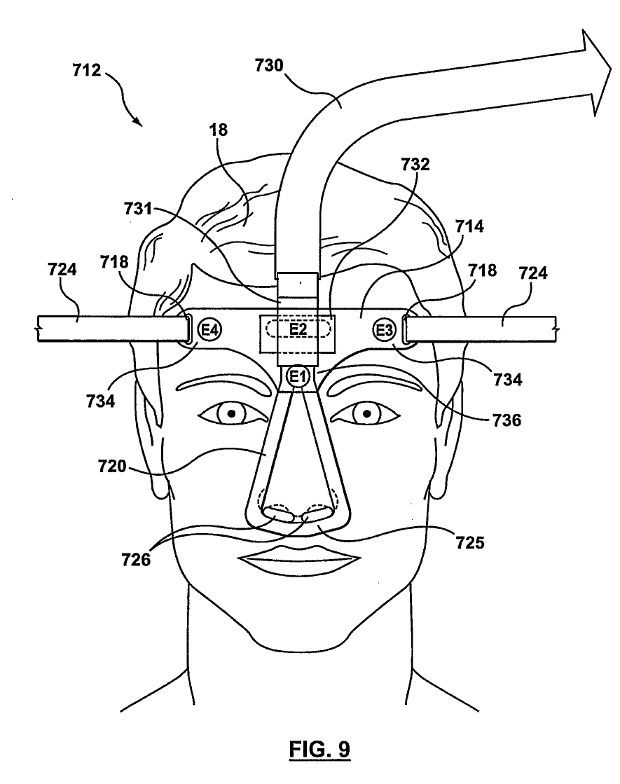

Referring now to FIG. 9, there is shown an alternative mask assembly 712. The mask assembly 712 comprises a flexible forehead plate 714 for holding electrodes E1, E2, E3 and E4 in position on the forehead of the wearer 18 and a strap 724 for securing the forehead plate 714 to the wearer 18. Mask assembly 712 also comprises a nasal interface 720 connected to forehead plate 714 via connector member 732. Nasal interface 720 receives pressurized gas through a gas supply tube 730 for feeding the gas directly into the nostrils of the wearer 18 through gas outlet ports 726 of nasal interface 720.

Nasal interface 720 is shown in FIG. 9 as being shaped as a loop extending downwardly over the wearer's face from the forehead and diverging around the nose. The divergent limbs of the loop of nasal interface 720 are joined at the bottom by a lip portion 725 which is designed to generally overlie the upper lip of the wearer 18 and be held upwardly against the wearer's nose so that outlet ports 726 feed directly into the nostrils of the wearer 18. Outlet ports 726 may be formed as tubular extensions which extend well into the wearers nostrils or may be formed so as to otherwise substantially occlude the wearer's nostrils so that relatively little of the gas supplied through gas outlets 726 leaks out of the nostrils.

Forehead plate 714 is formed roughly in a T-shape when viewed from the front while worn by the wearer 18. A lower portion 736 projects downwardly from forehead plate 714 and houses electrode E1 so as to generally, or at least partly, overlie the nasion area of the wearer 18. Electrodes E2, E3 and E4 are spaced laterally across forehead plate 714 in a similar manner to the arrangement shown in FIG. 2A. Electrode E2 acts as a ground electrode relative to the measured signals from electrodes E1, E3 and E4. Electrode E3 and E4 are positioned in laterally extending wings 734 of forehead plate 714 so as to overlie a central part of the forehead on each lateral side in a similar manner to the arrangements shown and described in relation to FIG. 2A.

Nasal interface 720 is connected to forehead plate 714 by connector 732 at a tubing portion 731 which extends between the nasal loop of nasal interface 720 and gas supply tube 730. The connection achieved by connector 732 may be mechanical or chemical, for example by snap fitting or adhesion. Other forms of removable or non-removable connection may be provided by connector 732.

Strap 724 is connected to forehead plate 714 at each lateral wing 734 by any suitable removable or non-removable attachment mechanism. As shown in FIG. 9, strap 724 is attached to forehead plate 714 by looping through a suitably shaped slot 718 in each lateral wing 734. Strap 724 passes around the head of wearer 18 and attaches to itself to form a loop snugly fitting around the wearer's head. The attachment of the parts of strap 724 together may be achieved by any suitable attachment mechanism.

Although not specifically shown in FIG. 9, forehead plate 714 may comprise additional sensors, such as those shown and described in relation to other embodiments. Additionally, the features of mask assembly 712 may be combined with, or substituted for, other features of other mask assembly embodiments shown and described herein, where such combination or substitution would result in a workable mask assembly. Features described in relation to other embodiments may be used instead of, or in addition to, the features of mask assembly 712, where such addition or substitution of features would result in a workable mask assembly.

As with other embodiments of the mask assembly, electrodes E1, E2, E3 and E4, as well as any other sensors located on forehead plate 714, may employ a wireless communication module to communicate with monitoring unit 16. Such a wireless communication arrangement is shown and described in co-pending U.S. patent application Ser. No. 11/130,221, filed on May 17, 2005 and entitled “Wireless Physiological Monitoring System” the entire contents of which is hereby incorporated by reference. Alternatively, dedicated conductors maybe connected to each such electrode or sensor and wired back to monitoring unit 16, for example along gas supply tube 730.

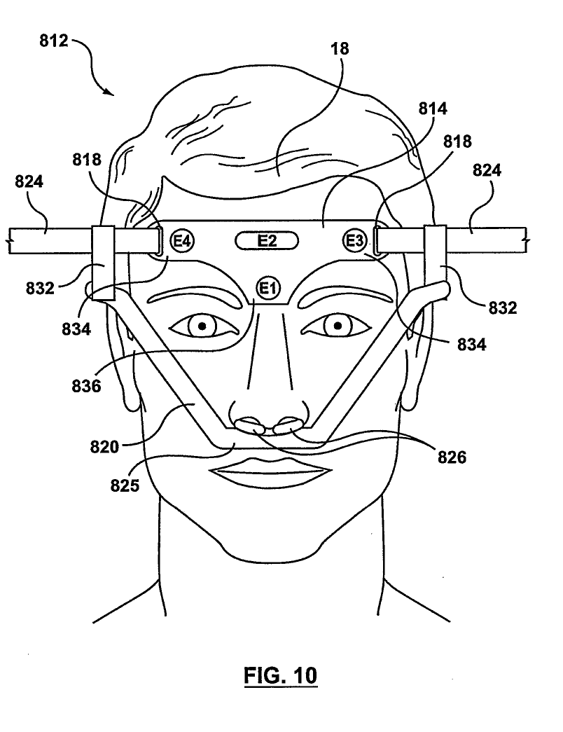

Referring now to FIG. 10, there is shown a mask assembly 812 according to another embodiment. Mask assembly 812 is similar to mask assembly 712, except that it uses an alternative nasal interface 820. Mask assembly 812 has a flexible forehead plate 814 and strap 824, which are the same as forehead plate 714 and strap 724, respectively. As mask assembly 812 is substantially similar to mask assembly 712, the same reference numerals are used to indicate the same features and functions as between the embodiments, except that the reference numerals in FIG. 10 all begin with an “8” in the hundreds column, as compared to the “7” in the hundreds column shown in FIG. 9. Because of these similarities, and in order to avoid repetition, we will only describe the features of the embodiment shown in FIG. 10 that are different to the features of the embodiment shown in FIG. 9.

Nasal interface 820 is of a slightly different form than nasal interface 720, whereby the gas supply tube (not shown) feeds into nasal interface 820 via a tubing loop that extends across the cheeks of the wearer and around to the back of the head or neck, rather than looping upwardly around the nose (as in FIG. 9). Nasal interface 820 is connected to forehead plate 814 via strap 824 using flexible connectors 832. Flexible connectors 832 serve to maintain lip portion 825 and gas outlets 826 in place against the wearer's nose by pulling up the tubing so that it passes above the ears or across the top of the ears of the wearer as it passes around to the back of the wearers head.

Forehead plates 714 and 814 are preferably formed using printed circuit sensors and electrodes, such as those supplied by Vermed, Inc. of Vermont, USA, under the trade name Pc-Sensor. Other forms of flexible printed circuit devices which may be used to form forehead plate 714 to 814 are made by Conductive Technologies, Inc. of York, Pa., USA. Alternatively, more conventional electrodes may be used within a flexible forehead plate formed of molded plastic, such as a polyvinyl chloride (PVC) plastic. Preferably, the plastic is relatively thin and flexible to accommodate the contours of the wearer's forehead, while having sufficient structural integrity and rigidity to maintain the electrodes in their respective positions and to enable suitable attachment of the straps 724, 824.

The nasal interface 720, which loops around the wearers nose, from across the central forehead, maybe of a form similar to that supplied by AEIOMed, Inc. of Minnesota, USA, based on its aura interface. A nasal interface of a kind similar to nasal interface 820 may be obtained from InnoMed Technologies, Inc., of Florida, USA based on their Nasal-Aire™ product line. It should be noted that, while FIGS. 9 and 10 show only one strap for securing the mask assembly to the wearer's head, additional straps may be used in a manner similar to the straps shown and described in relation to FIGS. 1, 2A, 2B, 3, 4 and 5. Also, if desired, one or more of the electrodes of mask assembly 712, 812 may be located on a part of the strap 724, 824 or on additional straps not shown.

Referring to FIGS. 11 to 14, embodiments of a device comprising a sensing unit for use in sensing electrical potentials for sleep stage determination are shown and described.

Referring in particular to FIG. 11, there is shown a sensing unit 1110 positioned on the forehead of a human head 110. Sensing unit 1110 has an electrode array including four electrodes E1, E2, E3 and E4 (labeled differently from the electrodes E1 to E4 described above in relation to FIGS. 1 to 10) formed thereon for overlying exposed skin surfaces of the forehead and nasion areas. Electrodes E1, E2, E3 and E4 are used to detect electrical potentials corresponding to EEG, EMG and EOG signals during a sleep study. Sensing unit 1110 generally functionally corresponds to forehead plates 714, 814 or forehead support member 214 (as shown in FIGS. 3, 4 and 5) in that it serves to locate electrodes at frontal positions on the wearer 18.

Sensing unit 1110 comprises a flexible plate-like member 1120 formed roughly in a T-shape when viewed from the front while worn on the head 110. A lower portion 1125 of flexible member 1120 projects downwardly from the substantially laterally extending body of flexible member 1120. Lower portion 1125 houses electrode E3 so as to be positioned to at least partly overlie the nasion area or an area adjacent thereto. Depending on the forehead structure of the head, electrode E3 may be positioned slightly above the nasion area, but generally on a centre line extending vertically through the forehead intermediate the eyes and eyebrows.

Electrodes E1, E4 and E2 are spaced laterally across sensing unit 1110. Electrode E4 acts as a ground electrode relative to the measured potentials from electrodes E1, E2 and E3. Electrodes E1 and E2 are positioned in laterally extending wings 1127 and 1128 located on respective right and left sides of the head 10 (as seen from the patient's perspective). Electrodes E1 and E2 and wings 1128 and 1127 are positioned widely (laterally) so that, for most forehead sizes and structures, the electrodes E1, E2 are respectively positioned on the forehead above and laterally beyond a vertical centerline through each eye. The greater lateral spacing of electrode E1 and E2 allows the sensing of a greater amount of relevant EEG data.

Ground electrode E4 is positioned generally centrally on sensing unit 1110 within a central area 1126 of flexible member 1120.

As shown in FIG. 11, flexible member 1120 has a connector limb 1132 extending from a left side (seen from the patient's perspective) thereof and a connector 1130 at an end of connector limb 1132. Connector 1130 is arranged to electrically couple conductors 1122 extending through sensing unit 1110 to a processing unit 1520 (shown and described in relation to FIG. 15), thereby forming an electrical connection between the processing unit and the electrodes E1, E2, E3 and E4 to which conductors 122 are electrically coupled.

According to some embodiments, sensing unit 1110 is formed mostly of flexible materials for placement on a forehead structure and for generally conforming to the shape of the forehead structure. Certain parts of sensing unit 1110 (for example, those around the electrodes) may have an adhesive substance, such as a foam adhesive layer, on an underside thereof, for affixing the sensing unit 1110 to the forehead prior to conducting the sleep study. Flexible circuitry, comprising conductors 1122, extends through sensing unit 1110 from each of the electrodes E1, E2, E3 and E4 to connector 1130. Thus, sensing unit 1110 can be used with forehead structures of varying shapes and sizes due to its flexibility and ability to conform and adhere to such varying forehead structures, as required.

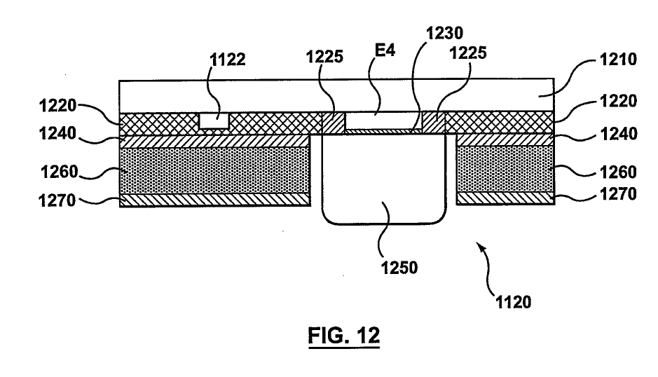

Sensing unit 1110 is shown in FIG. 12 in partial cross-section, taken along line A-A of FIG. 11. Flexible member 1120 employs a substrate 1210 of a flexible material such as a medical grade polyester film (or other material having similar properties). Substrate 1210 forms the top (or upper or outer) layer facing away from the forehead. Substrate 1210 has sufficient rigidity to form the base for flexible circuitry to be printed (or otherwise formed) thereon and enable subsequent conductive and insulating layers to be formed thereon, while having sufficient flexibility to enable the entire flexible member 1120 to bend to generally conform to the shape of the forehead to which it is to be affixed.

The substrate 1210 may be about 3 to 8 thousandths of an inch thick, for example. Adhesive 1270 is of a relatively weak strength and is used to affix at least a part of the flexible member 1120 to the skin of the forehead. Adhesive 1270 is provided on a layer of medical grade adhesive foam 1260 of approximately 1/32 of an inch thickness. The foam 1260 is adhered to an insulation layer 1220 on the substrate 1210 on one side with a relatively strong adhesive 1240 and has adhesive 1270 on the opposite side for removable attachment to the test subject. Insulation layer 1220 is applied directly to the substrate 1210 to insulate electrical conductors and is formed of an appropriate epoxy or resin. The electrodes E1 to E4 may comprise a silver or silver chloride layer formed on the substrate 1210. The substrate 1210 has flexible circuit tracings formed thereon for constituting the conductors 1122 between electrodes E1 to E4 and output connector 1130. Such circuit tracings may comprise silver and preferably have a dielectric layer (such as insulation layer 220) formed thereover.

Prior to affixation to the forehead, sensing unit 1110 may have backing sheets (not shown) on those parts of sensing unit 1110 that have an adhesive substance 1270 on their undersides for adhesion to the skin. Each such backing sheet is removed immediately prior to adhesion of the relevant part of sensing unit 1110 to the corresponding forehead area or areas. For electrodes E1 to E4, an area of conductive gel (not shown), such as hydrogel, is interposed between the respective electrode and the skin surface (instead of the adhesive foam 1260), for facilitating conductivity of electrical signals between the electrodes E1 to E4 and the skin.

Sensing unit 1110 is a generally flat device, as viewed from the user's perspective, prior to affixation to the test subject. However, sensing unit 1110 does have several layers, as described above. In use of sensing unit 1110, and with the backing sheets removed, the adhesive foam 1260 and electrodes E1 to E4 are positioned to lie against the skin. These skin contact surfaces may be conveniently referred to as being formed on the underside of the sensing unit 1110. Printed labeling, including affixation instructions, may be provided on the side of sensing unit 1110 that does not contact the skin.

Electrodes E1 to E4 are formed on substrate 1210, either directly or on a thin priming or separation layer (not shown) coating the underside of substrate 1210. Electrodes E1 to E4 are electrically coupled to output connector 1130 via conductors 1122 in the form of flexible circuit tracings formed on substrate 1210. As with electrodes E1 to E4, conductors 1122 may be directly formed on substrate 1210 or may be separated therefrom by a priming or separation layer. Portions of flexible member 1120 that are not to be exposed to the forehead (such as conductors 1122) are covered by insulation layer 1220.

In the embodiment shown in FIG. 12, electrode E4 comprises a silver chloride layer 1230 on its outer face for facilitating conductivity with the skin via a conductive gel in contact with electrode E4. The conductive gel is provided as a liquid hydrogel and is impregnated into a porous foam sponge 1250 that contacts the skin when the sensing unit 1110 is positioned on the patient's forehead. Sponge 1250 is adhered to substrate 1210 by an adhesive layer 1225 disposed around the electrodes. In order to allow for compression of the sponge during skin contact, a gap may be formed on either side of the sponge 1250 between the sponge 1250 and the foam layer 1260.

In an alternative embodiment, a substantially more viscous conductive gel can be used instead of the sponge 1250 and liquid hydrogel, in which case, the adhesive layer 1225 and the compression gap are not required. The above impregnated sponge arrangement and the viscous hydrogel arrangement are both commercially available from Vermed, Inc. of Bellows Falls, Vt., USA.

Adhesive layer 1270 and conductive sponge 1250 may be covered by the protective backing sheet or layer (not shown) so that the adhesive and conductive qualities of the adhesive layer 1270 and conductive sponge 1250 are preserved until application of flexible member 1120 to the forehead. The total thickness of sensing unit 1110, including substrate 1210, may be in the range of 0.7 to 1.5 millimeters, approximately.

The embodiment shown in FIG. 12 is not to scale, is for purposes of illustration only and some variations or modifications may be made, depending on the specific requirements of the sensing unit embodiment and methods of forming it.

While the sensing unit embodiments shown and described herein generally have a unitary flexible member including two wings and a projecting portion, alternatively each of the areas or portions of the sensing unit having electrodes may be formed on a separate, but connected, substrate.

In an alternative embodiment of sensing unit 1110, metallic disk electrodes may be used with a flexible member formed of molded plastic, such as a polyvinylchloride (PVC) plastic. In such an embodiment, the plastic is preferably relatively thin and flexible to accommodate the contours of the wearer's forehead, while having sufficient structural integrity and rigidity to maintain the electrodes in their respective positions. Such a molded plastic flexible member may be shaped similarly to flexible member 1120 and may employ a suitable adhesive to secure it in place on the forehead. Alternatively, or in addition, a strap or other mechanical means may be used to secure the sensing unit 1110 in place on the wearer's forehead.

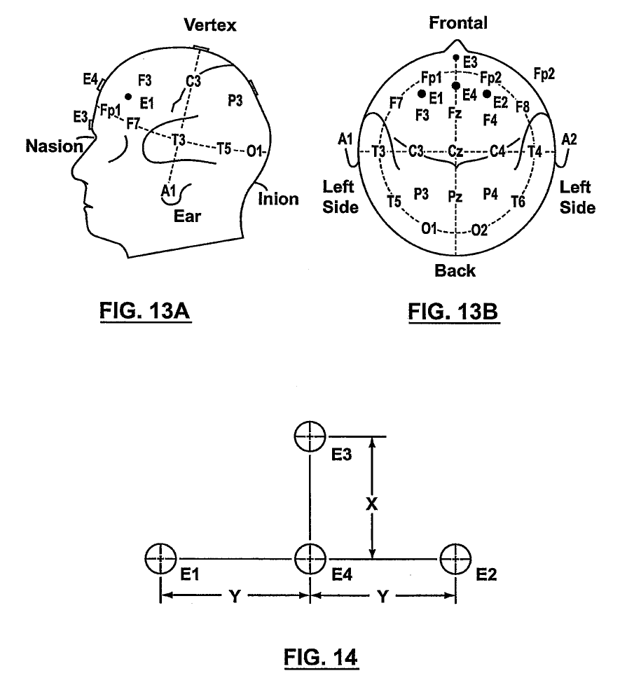

Referring in particular to FIGS. 13A, 13B and 14, the positioning of electrodes E1, E2, E3 and E4 is described in further detail. FIGS. 13A and 13B indicate the likely positions of electrodes E1 to E4 on a human head, relative to the standard 10-20 electrode positions. As can be seen from FIGS. 13A and 13B, reference electrode E3 is positioned adjacent the nasion area. Electrode E3 is located on flexible member 1120 so that, for most forehead structures, it will be positioned immediately above the nasion and in between the eyebrows. Electrode E3 is thus positioned on the vertical centerline of the head in a position lower than the line extending laterally through frontal positions Fp1 and Fp2.

Electrode E4 is positioned on the midline (vertical centerline) below frontal position Fz but above the lateral frontal line extending through frontal positions Fp1 and Fp2. Electrodes E3 and E4 are separated by a distance X, as shown in FIG. 14, where X may be about 35 to 55 mm. In one embodiment, X may be about 40 to 50 mm. In a further embodiment, X may be about 44 mm.

As shown in FIG. 14, electrodes E1 to E4 are arranged in a T-shaped configuration, with reference electrode E3 at a bottom of the T and electrodes E1, E2 and E4 forming the top line of the T. In alternative embodiments, the electrode configuration need not be strictly T-shaped. For example, ground electrode E4 may be shifted up or down so that it is not strictly in line with electrodes E1 and E2.

Further, electrodes E1, E2 and E3 are arranged in a triangular configuration, where the distance between electrodes E1 and E3 is the same as the distance between electrodes E2 and E3, but is not the same as the distance between electrodes E1 and E2. Thus, electrodes E1, E2 and E3 are arranged in an isosceles triangular configuration. This configuration allows the electrodes to be arranged in sensing pairs E1-E3 and E2-E3 to sense EEG, EOG and EMG potentials, while sensing electrode pair E1-E2 is also arranged to sense EEG, and EOG potentials. The E1-E3 and E2-E3 electrode pair orientations may be configured to be substantially orthogonal to each other.

Electrodes E1 and E2 are each laterally separated from electrode E4 by a distance Y that may be the same as distance X or may be different therefrom. The total distance (2Y) between electrodes E1 and E2 is, according to one embodiment, between about 70 and 110 mm. In another embodiment, the separation of electrodes E1 and E2 is about 80 to 100 mm. In a further embodiment, the separation is about 90 mm.

Electrodes E1 and E2 are located on flexible member 120 so as to be positioned on the forehead at forehead locations above and laterally beyond standard frontal positions Fp1 and Fp2, respectively. This wider and higher spacing of electrodes E1 and E2 across the frontal area allows for a greater range and quality of EEG potentials to be detected than if the standard Fp1 and Fp2 positions were used. This greater range can be used to compensate for the lack of a reference electrode positioned at A1 or A2 behind the ear.

The described configuration of electrodes E1, E2 and E3 allows for simultaneous sensing of EEG, EOG and EMG potentials using a single electrode assembly on a flexible member that is easily applied by a patient to his or her own forehead prior to self-initiation of the sleep study. Thus, sensing device 1110 is easily applied in a home setting without the need for the patient to be studied in an artificial environment and without the need for a medical technician to affix the electrodes to the patient's head 110.

In alternative embodiments of sensing unit 1110, one or more of electrodes E1 to E4 may comprise a needle electrode specifically configured for EMG potential detection. Alternatively, or in addition, one or more of electrodes E1 to E4 may have a wireless transmitter associated therewith (instead of a conductor 1122) for transmitting wireless signals to a nearby receiver, such as is described in U.S. patent application Ser. No. 11/130,221, entitled “Wireless Physiological Monitoring System”, filed May 17, 2005, the entire contents of which is hereby incorporated by reference.

Although not shown in FIG. 11, embodiments of sensing unit 1110 may have a strap attachable to each lateral wing 1127, 1128 for securing sensing unit 1110 to the head. Such a strap may be in addition or alternative to adhesive 1270 for securing sensing unit 1110 in place. In place of a strap, other means for securing the sensing unit to the head may be employed.

In further embodiments, electrodes E1 to E4 are removably attachable to flexible member 1120. In such embodiments, electrodes E1 to E4 are formed as metallic disk electrodes that have male snap connector parts on a back surface thereof for engaging a corresponding female snap connector part positioned on flexible member 1120. In such embodiments, conductors 1122 are electrically coupled to the female snap connector parts, which form a mechanical and electrical connection with the electrodes via the male snap connector parts on each electrode.

In such embodiments, the underside of flexible member 1120 may not employ an adhesive to affix the flexible member 1120 to the forehead. Rather, a strap or band may be used to secure the flexible member 1120 in the appropriate location. In order to affix the electrodes E1 to E4 to the appropriate locations on the forehead and nasion areas, each electrode may be provided with a portion of adhesive foam around the outside of the conductive contact surface of the electrode. Alternatively, the conductive gel on the contact surface of the electrodes may have sufficient adhesive properties to obviate the use of adhesive foam portions around the electrodes.

The removably attachable electrode embodiment allows the flexible member 1120 to be reusable while the electrodes can be disposed of after each use. In this embodiment, the flexible member 1120 may be comprised of a material having greater flexibility and/or deformation properties than the polyester film or PVC described above. A suitable material may comprise a cloth or other woven material. Alternatively, the flexible member 1120 may be comprised of a relatively more rigid material, such as PVC, although this rigidity is not strictly required if each electrode is held in place on the skin by the portion of adhesive material surrounding it.

While sensing unit 1110 is described in relation to use in sleep stage determination, the sensing unit 1110 can be usefully applied in combination with other apparatus or software to record other results of diagnostic significance. Examples of such other apparatus include mask assemblies for providing positive airway pressure (PAP) to the patient, such as is described above in relation to FIGS. 1 to 10. Embodiments may also be used within the context of an intensive care unit (ICU), for example to assist in detection of a seizure, stroke, ischemia, burst-suppression or brain hemorrhage or for use in determining a level of consciousness, sedation or delirium of a patient.

According to alternative embodiments, additional sensors, which may be electrodes or other forms of sensors, may be provided for positioning at other locations on the head. For example, an additional electrode may be placed behind or in front of the ear or ears, for use as an active or reference electrode. Such additional sensors may be coupled (for example, on a unitary substrate) to flexible member 1120 for electrical connection to the processing unit via connector 1130. Alternatively, a separate connector and/or substrate may be used for electrically coupling the additional sensor or sensors to the processing unit.

While certain embodiments described herein contemplate the use of four electrodes E1 to E4 located on the flexible member 1120, for each of those four electrodes, more than one electrode may be used in place of the single electrode. In still further embodiments, the sensing unit 1110 may employ more than four electrodes at various positions on the flexible member 1120. In a further alternative embodiment, the ground electrode E4 may be omitted or its position varied.

While the configuration of the electrode array of sensing unit 1110 is shown arranged in a T-shaped configuration, alternative configurations, for example where the central ground electrode E4 is positioned higher or lower, may be employed. However, electrode configurations that necessitate placement of one of the electrodes over a hair-covered part of the scalp or forehead are less desirable than those that allow placement of the electrodes over hairless areas of the scalp or forehead. Thus, shapes analogous to a T-shape, such as a cross-shape, Y-shape or other shapes having laterally extending wings and a downwardly projecting portion, may be employed to a similar effect to the embodiments using a T-shaped electrode configuration on the flexible member. In some embodiments, the lateral wings of the flexible member 1120 may extend further laterally and droop down, in a shape similar to ram's horns, to cover the temple areas on either side of the head. This allows additional electrodes to be placed over the temple areas for increased EEG sensing capability.

Referring now to FIGS. 15 to 19, embodiments of a system and method for use in processing measured electrical potentials corresponding to biological signals for sleep stage determination are shown and described. These embodiments employ the frontal electrode array of the embodiments of sensing unit 1110, forehead plate 714, 814, forehead support member 214 or mask assembly 12 described above.

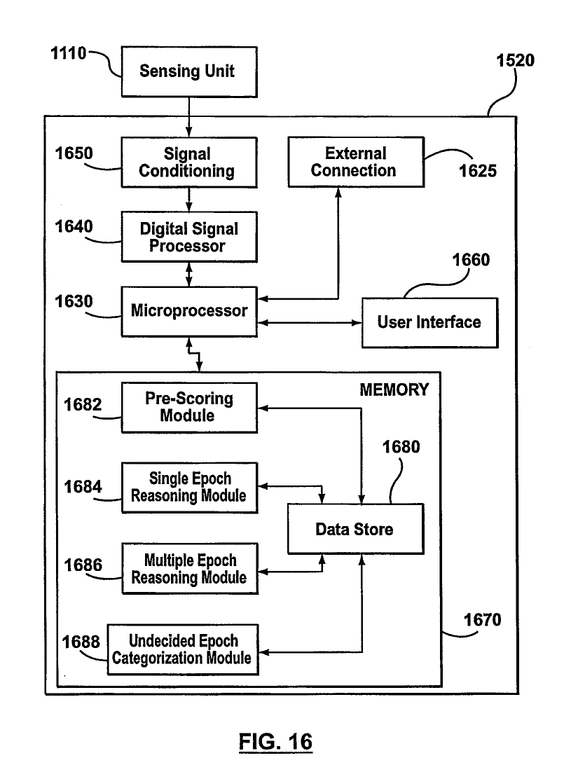

Referring in particular to FIG. 15, there is shown a system 1500 for sleep stage determination including the sensing unit 1110 (as one example of a forehead member comprising the frontal electrode array) and a processing unit 1520 in communication with, and coupled to, sensing unit 1110. Processing unit 1520 accepts electrical potentials from sensing unit 1110 as input, transforms the received electrical potentials into suitable biological signal data and performs digital signal processing on the biological signal data. Processing unit 1520 may also accept instructions via a user interface 1660 (FIG. 16) or provide feedback related to operation of system 1500. Processing unit 1520 may further communicate over a network 1560 with a server system 1570 in order to, for example, exchange data or instructions. An example embodiment of processing unit 1520 is shown in more detail in FIG. 16.

Network 1560 may comprise a suitable computer or telephone network, such as a local area network (LAN). Other networks, such as a wireless local area network (WLAN), the public Internet, or a public switched telephone network (PSTN), may also form part of network 1560.

Server system 1570 may be used to provide various facilities better suited to a centralized system, such as: storage of patient records; storage of sleep data; management of remote processing units; downloading updated software to procession unit 1520; facilities for communicating other data, such as user instructions or administrative commands, to remote devices; and facilities for receiving data, such as user queries or diagnostic information, from remote devices. In one embodiment, server system 1570 may be comprised of a plurality of physical computers, not necessarily co-located.

Referring in particular to FIG. 16, processing unit 1520 is shown in further detail. Processing unit 1520 contains elements required for processing the electrical potentials captured by sensing unit 1110. Electrical potentials are received from sensing unit 1110 and undergo signal conditioning using a signal conditioning module 1650 to transform the received electrical potentials into suitable biological signal data. Such signal conditioning may include filtering signals in the received data into various frequency bands, as well as amplification and removal of any DC offset.

Conditioned signals are supplied to a digital signal processor 1640 for analysis under the control of, or in combination with, a microprocessor 1630. Processed biological signal data is stored in a memory 1670 by microprocessor 1630. Microprocessor 1630 retrieves stored data from memory 1670 as needed, for example to provide output or perform further processing. Microprocessor 1630 also transmits data to user interface 1660, for example, to generate a display to a user of processing unit 1520. Additionally, microprocessor 1630 may receive operational instructions from a user via user interface 1660.

Signal conditioning module 1650 may comprise electronic circuitry on an application-specific integrated circuit (ASIC) designed for specific signal conditioning purposes, including amplification, removal of any DC offset, analog to digital signal conversion and filtering signals into various frequency bands. Alternatively, commercially available discrete components may be used to perform each function. Alternatively, a suitable combination of custom and commercial components may be used to perform the signal conditioning.

Digital signal processor (DSP) 1640 may be a suitable commercially-available DSP, general purpose microprocessor, application specific integrated circuit (ASIC), field programmable gate array (FPGA), or a multiple or combination of any of these devices and is used to perform various calculations that require vector processing of data, such as Fast Fourier Transform (FFT) operations. In an alternative embodiment, a single module may perform the signal conditioning and DSP functions.

Microprocessor 1630 may be a suitable commercially-available DSP, general purpose microprocessor, ASIC, FPGA, or a multiple or combination of any of these devices and is used to perform all computation and control functions not performed by other elements of the system, such as conditional branch evaluation, data input/output and device control.

User interface 1660 consists of one or more input or output devices for human interaction, such as a keyboard, touchpad, printer or visual display. User interface 1660 also comprises the output elements required to communicate data and command options to a user, such as forms, tables, buttons and other appropriate elements. User interface 1660 may be adapted to accommodate a variety of uses or patients, for example to provide auditory or Braille output.

Microprocessor 1630 may also communicate bi-directionally with an external connection 1625. External connection 1625 may comprise a wireless communication interface or a wired communication interface for communication with a remote device or system over, for example, network 1560. Alternatively, external connection 1625 may employ a standard communications interface, such as a Universal Serial Bus (USB), to communicate with an auxiliary or peripheral device to enable added functionality. Additionally, external connection 1625 may also connect directly to another processing unit 1520 or server system 1570.

Microprocessor 1630 reads, writes and otherwise manipulates data in memory 1670. The contents of memory 1670 may contain both biological signal data and operational instructions associated with a computer program to be used in evaluating the signal data. Memory 1670 may be composed of both volatile and non-volatile memory components, including solid state, magnetic or optical storage, such as flash programmable memory and hard disk drives, or a combination thereof. In addition, future memory technologies may be employed as they become available and where they provide equivalent or enhanced functionality.

Several computer program modules are stored concurrently in memory 1670, including: a pre-scoring module 1682 for quickly categorizing easily-identified sleep stages; a single epoch reasoning module 1684 for identifying sleep stages capable of being recognized within a single observation interval; a multiple epoch reasoning module 1686 for identifying sleep stages which require signal observation over a multiplicity of observation intervals; and an undecided epoch categorization module 688 for categorizing previously uncategorized epochs. Each module may exist both as computer program instructions and as a computational representation of its current processing state.

Each of modules 1682, 1684, 1686 and 1688 is contained within memory 1670 and may access and update the data store 1680 as required, to update signal data and contextual information, receive updated signal data and contextual information, or otherwise read or manipulate relevant data. Contextual information includes sleep stage information as well as information regarding changes in the parameters used to determine a sleep stage. Contextual information may be combined with signal data to categorize an epoch as belonging to a particular sleep stage. For example, if the current epoch follows sleep stage1, AND the Betal power increases more than 50%, AND the spindle activity is not high, then the current epoch is scored (categorized) as Wake.

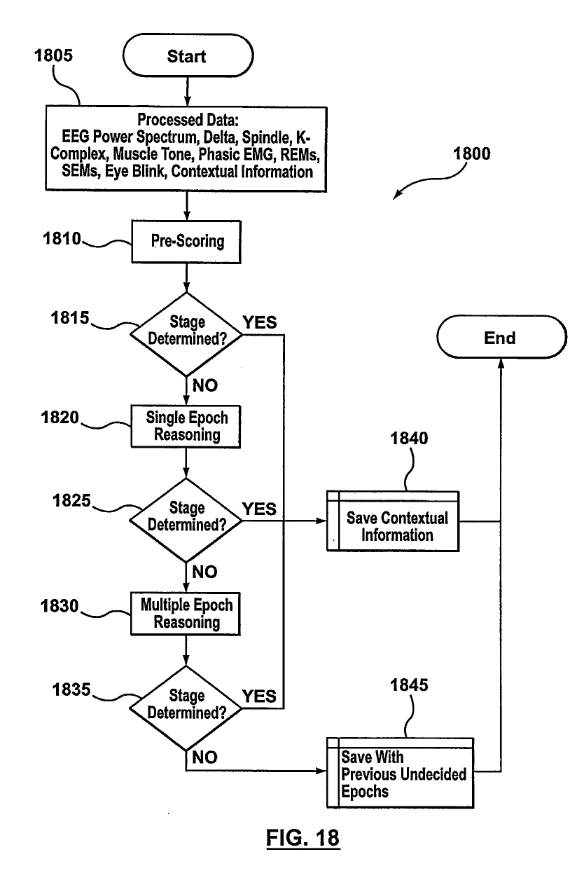

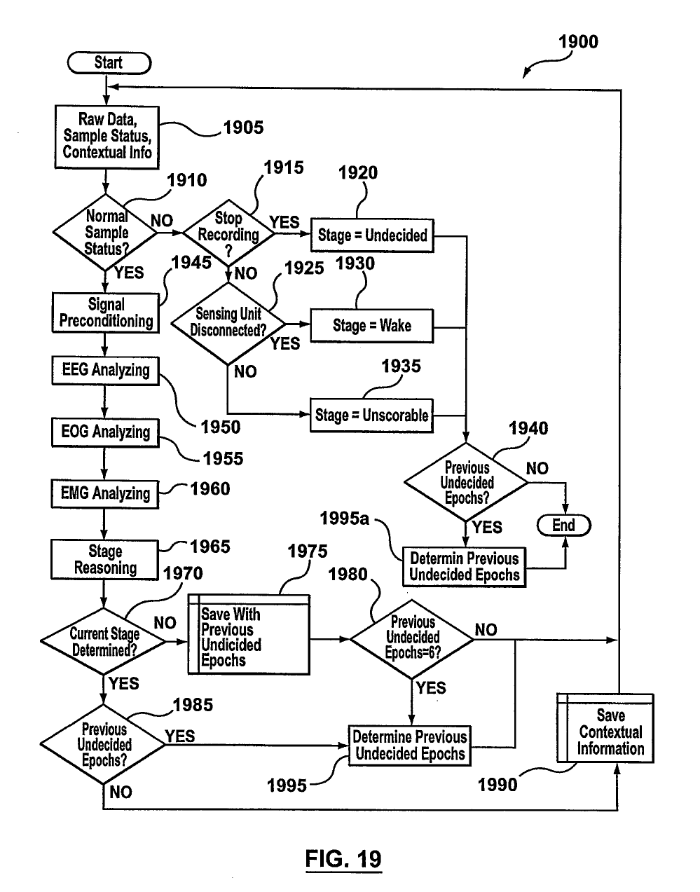

The functions of modules 1682, 1684, 1686 and 1688 may be further subdivided or supplemented with additional modules, for example to increase processing capacity. Additional detail regarding the function of modules 1682, 1684, 1686 and 1688 is provided below, particularly in paragraphs describing FIGS. 18 and 19 and in pseudo-code describing software operation.

One or more of the above elements, including signal conditioning module 1650, digital signal processor 1640, microprocessor 1630, memory 1670 and external connection 1625, may be combined into a single physical device, for example, a field programmable gate array (FPGA).

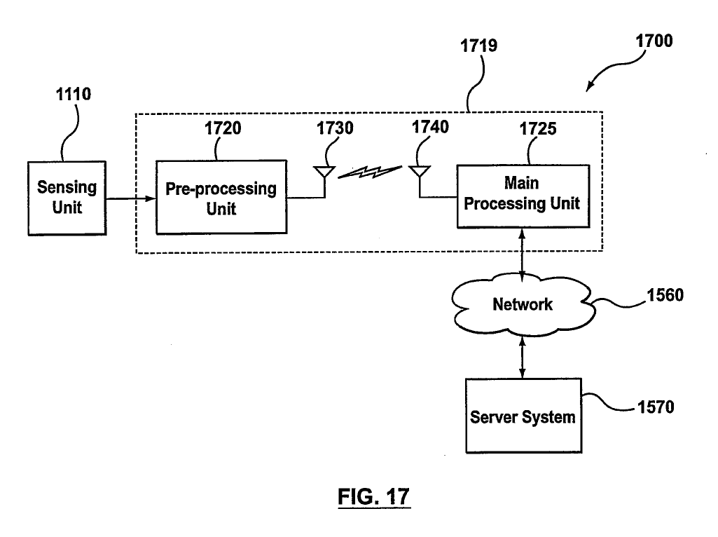

In an alternative embodiment, processing unit 1520 may be subdivided into component units, for example, a pre-processing unit and a main processing unit, such as is shown in FIG. 17. Such an arrangement would allow for one main processing unit to service one or more pre-processing units, which may be helpful in certain clinical settings.

Referring in particular to FIG. 17, there is shown a system 1700, comprising: sensing unit 1110; a distributed processing unit 1719 comprising a pre-processing unit 1720, wireless communication interfaces (including transceivers) 1730 and 1740 and a main processing unit 1725; network 1560; and server system 1570. Distributed processing unit 1719 is functionally equivalent to processing unit 1520, but possesses certain features, such as wireless operation and a many-to-one relationship of pre-processing units to main processing unit, that may make it more suitable for particular applications.

In this embodiment, sensing unit 1110 is coupled (via connector 1130) to pre-processing unit 1720, which performs a subset of the functions, for example, signal conditioning and digital signal processing, performed by processing unit 1520. In this embodiment, sensing unit 1110, pre-processing unit 1720 and wireless interface 1730 effectively form a sub-system that can be worn by the patient without needing to be physically connected to, or co-located with, the main processing unit 1725.

Pre-processing unit 1720 uses wireless communication interface 1730, which may employ a low-power, short-range antenna and a suitable wireless communication protocol, to communicate with wireless communication interface 1740. Wireless communication interface 1740 is connected to main processing unit 1725, which performs the remainder of the functions of processing unit 1520 not performed by pre-processing unit 1720. One main processing unit 725 may communicate with a plurality of pre-processing units 1720.

Main processing unit 1725 may further communicate over network 560 with server system 1570. In another alternative embodiment, both pre-processing unit 1720 and main processing unit 1725 may communicate directly with each other and/or with server system 1570 over network 1560.

Wireless communication interfaces 1730 and 1740 employ standard commercially-available hardware and operate over portions of the electromagnetic spectrum using common networking standards, for example the IEEE 802.11, Bluetooth or IrDA family of protocols. In an alternative embodiment, wireless communication interfaces 1730 and 1740 may be of a custom design to enhance certain characteristics, such as low power or secure operation, to suit the particular application of system 1700. Future networking protocols and interfaces, possibly operating in other areas of the electromagnetic spectrum, may be substituted as they become available, where suitable.

In an alternative embodiment, for example, in a clinical or hospital environment, wireless operation may not be desirable or necessary. Therefore, wired communication interfaces, such as members of the IEEE 802.3 or 1394 families, may be used in place of wireless communication interfaces 1730 and 1740.

Collection of electrical potentials corresponding to biological signal data by sensing unit 1110 occurs continuously over a period of a number of hours. At predetermined intervals, called epochs, an evaluation process is invoked to evaluate or categorize collected data. In an alternative embodiment, epoch frequency may be varied, for example, to increase the rate of data collection during periods of high activity.

Referring in particular to FIG. 18, there is shown a flow chart illustrating an evaluation process 1800, which is an embodiment of one iteration of the process invoked for an epoch by processing unit 1520 or main processing unit 1725 to categorize collected signal data as belonging to a particular stage of sleep. Processed data, for example, consisting of EEG power spectrum, delta, spindle, K-complex waves, muscle tone, phasic EMG, rapid eye movements (REMs), slow eye movements (SEMs), eye blinks and other contextual information, is gathered at step 1805 for pre-scoring evaluation at step 1810.

Upon completion of pre-scoring step 1810, a test is performed at step 1815 to check if the sleep stage has been determined, based on the pre-scoring. If the sleep stage is determined, contextual information is saved at step 1840 and process 1800 ends. If the sleep stage cannot yet be determined, the evaluation process continues to single epoch reasoning, at step 1820.

Upon completion of step 1820, a test is performed at step 1825 to check if the sleep stage has been determined, based on the single epoch reasoning. If the sleep stage is determined, contextual information is saved at step 1840 and process 1800 ends. If the sleep stage cannot yet be determined, the evaluation process continues to multiple epoch reasoning, at step 1830.

Upon completion of step 1830, a test is performed at step 1835 to check if the sleep stage has been determined, based on the multiple epoch reasoning. If the sleep stage is determined, contextual information is saved at step 1840 and process 1800 ends. If the sleep stage cannot yet be determined, the data is saved as an undecided epoch, at step 1845, and process 1800 ends. An undecided epoch is equivalent to an unscored epoch or undetermined epoch.

Processed data gathered at step 1805 is the collection of data obtained by sensing unit 1110 and further conditioned and categorized by one or more of signal conditioning module 1650, digital signal processor 1640 and microprocessor 1630. Processed data gathered at step 1805 is stored in memory 1670 in a format suitable for further evaluation.

Pre-scoring step 1810, which is performed by microprocessor 1630 executing pre-scoring module 1682, evaluates processed data 1805 to identify patterns that are easily categorized, for example, such as certain characteristics consistent with a waking stage. Upon evaluation of various pre-scoring rules, such as those described below in pseudo-code, it is determined at step 1815 whether the sleep stage can be categorized by the pre-scoring process. If the pre-scoring step was successful at determining the sleep stage, the determined sleep stage is assigned to the epoch under consideration, contextual information is saved at step 1840 and the current iteration ends. If no sleep stage has been determined, the process continues to single epoch reasoning at step 1820.

Pseudo-code describing the decision process of one embodiment of pre-scoring step 1810 is shown below. A glossary of acronyms and abbreviations used in the pseudo-code is provided in Table 3 below.

Pre-Scoring

| | |
|---|---|
| IF (AftM \|\| AftW) | |
| IF (Noisy \|\| MA > 12 \|\| FEMs >= 6 \|\| (AlpPk && BSIHi)) | Cstage = W |
| ELSE IF (AlpEEG && (ASI >1.2 \|\| FEMs >= 3 \|\| BSI Decrs < 70%)) | |
| OR (BtaEEG && (ASI > 0.6 \|\| FEMs >= 3 \|\| Tht Pwr Low)) | |
| IF (FstWv Pwr VH && BSIHst && (FSPLow \|\| AlpPwrHi \|\| BSI | |
| Incrs > 20%)) | Cstage = W |
| ELSE IF (FEMs >= 6 && BSIHi && ASI >= 0.6 && FSPLow) | Cstage = W |
| ELSE IF (MA > 15) | Cstage = MT |
| IF (Cstage == MT or Cstage == W) | |
| IF (AftR_W or AftR_M) | Cstage = W |
| ELSE IF (AftR && Cstage == MT) | Cstage = R_T |
| ELSE IF (AftR && Cstage == W) | Cstage = R_W |
| ELSE CONTINUE | |

Single epoch reasoning step 1820, which is performed by microprocessor 1630 executing single epoch reasoning module 1684, evaluates processed data 1805 to identify patterns that are capable of being categorized using information obtained only from the epoch that is currently being analyzed, for example such as REM sleep or transitions between REM sleep and another sleep stage. Upon evaluation of various single epoch reasoning rules such as those described below in pseudo-code, a decision is made at step 1825. If the single epoch reasoning step is successful at determining the sleep stage, the determined sleep stage is assigned to the epoch under consideration, contextual information is saved at step 1840 and the current iteration ends. If no sleep stage has been determined, the process continues to multiple epoch reasoning at step 1830.

Pseudo-code describing the decision process of one embodiment of single epoch reasoning step 1820 is shown below. A glossary of acronyms and abbreviations used in the pseudo-code is provided in Table 3 below.

Single Epoch Reasoning

| | |
|---|---|
| IF (!AftW && !REMBgrd && !Wakening && !AftR && !BSIHst && Delta > 20%) | |
| IF (AftDS \|\| DelPwr VH) | Cstage = SD |
| ELSE | Cstage = S2 |
| ELSE IF (!AftW && !AftDS && MslTLow && MA < 3 && Spindle not | |
| high && ATI < 0.4) | |
| IF (FstWv Pwr Low && FSPLwst && BSIHst) | |
| IF (BSI Incrs > 20% \|\| AftS2) | Cstage = REM |
| ELSE IF (AlpPwrLow && REMs > 0 && BSIHi && Spindle not | |
| found) | |
| IF (AftR && BSI Decrs < 50% && FSP lncrs < 200%) | Cstage = REM |
| ELSE IF (!AftR && (BSILow \|\| FSPHst)) | |
| IF (AftS2 \|\| S2Wvs) | Cstage = S2 |
| ELSE | Cstage = S1 |
| ELSE IF (S2Wvs) | Cstage = R_S2 |
| ELSE | Cstage = R_S1 |
| ELSE IF (REMBgrd) | |
| IF (!S2Wvs && MA < 6 && AftR) | Cstage = REM |
| ELSE IF (S2Wvs) | Cstage = R_S2 |
| ELSE IF (MslTVH \|\| AlpPk) | Cstage = R_M |
| ELSE | Cstage = R_S1 |
| ELSE IF ((AftR \|\|AftRLike) && MA < 6 && Spindle not high && | |
| AlpPwrLow && !BSILow) | |
| IF (AlpPk \|\| MslTVH) | |
| IF (AftR) | Cstage = R_M |
| ELSE IF (AftR_M or AftR_W) | Cstage = W |
| ELSE IF (! AlpPk && HBSI > 6 && LFSP > 6) | |
| IF (!MslTLow && EEGPwr VH && REMs = 0) | Cstage = R_M |
| ELSE IF (BSIHst \|\| (MslTLow && REMs > 0) \|\| (MslTLow && | |
| FSPLwst)) | Cstage = REM |
| ELSE IF (S2Wvs) | Cstage = R_S2 |
| ELSE IF (AftR && (AlpPwrHi \|\| FstWv Pwr High)) | Cstage = R_W |
| ELSE | Cstage = R_S1 |
| ELSE IF (!S2Wvs && MA < 1 && (AftR \|\| AftRLike) && ATILow && | |
| EEGPwr Low && FstWv Pwr Low) | |
| IF (BSIHi && BSI Decrs < 50% && (FSPLow \|\| FSP Incrs < 200%)) | Cstage = R_S1 |

| | |
|---|---|
| IF (Cstage == UNSCORED) | CONTINUE |
| ELSE | STOP |

Multiple epoch reasoning step 1830, which is performed by microprocessor 1630 executing multiple epoch reasoning module 1686, evaluates processed data 1805 to identify patterns that may require data from multiple epochs to categorize sleep stage, for example sleep stages S1 or S2. Upon evaluation of various multiple epoch reasoning rules such as those described below in psedo-code, a decision is made at step 1835. If the multiple epoch reasoning step is successful at determining the sleep stage, the determined sleep stage is assigned to the epoch under consideration, contextual information is saved at step 1840 and the current iteration ends. If no sleep stage has been determined, the current epoch is categorized as undecided, data associated with the undecided epoch is saved at step 1845 and the current iteration ends.

Pseudo-code describing the decision process of one embodiment of multiple epoch reasoning step 1830 is shown below. A glossary of acronyms and abbreviations used in the pseudo-code is provided in Table 3 below.

Multiple Epoch Reasoning

| | |
|---|---|
| IF (AlpPk && !SpnPk && Spindle not found && BSIHst) | |
| IF (! AlpPwrLow && ( AlpPwr or FstWv Pwr Incrs > 20%) ) | Cstage = W |
| ELSE | Cstage = S1 |
| ELSE IF ((AftW \|\| AftM) && AlpPwrLow && AlpPwr Decrs > 40% && | |
| FEMs < 3) | |
| IF (S2Wvs) | Cstage = S2 |
| ELSE | Cstage = S1 |
| ELSE IF (!AftW && !MslTLow && BSIHst \|\| FstWv Pwr VH && | |
| (EEGPwr \|\| FstWv Pwr VH) && !FSPHst) | |
| IF (AftS1 or AftR && Bta1Pwr Incrs > 50%) | |
| IF (Spindle not high) | Cstage = W |
| ELSE IF (AlpPwr Incrs > 50%) | |
| IF (MslTVH) | Cstage = MT |
| ELSE | Cstage = W |
| ELSE | Cstage = S1 |
| ELSE IF (AftS2 \|\| AftDS && Bta2Pwr Incrs > 100% && BSI VH) | |
| IF (FstWv Pwr High && Spindle not high) | |
| IF (MA > 2 \|\| FSPHi) | Cstage = MT |
| ELSE | Cstage = W |
| ELSE IF (Spindle not high && S2Wvs) | Cstage = S2 |
| ELSE | Cstage = S1 |
| ELSE IF (BSI VH && FSPLwst) | |
| IF (FstWv Pwr High && AftW or AftM) | Cstage = W |
| ELSE IF (EEGPwrVH) | Cstage = MT |
| ELSE IF (AlpPwrLow) | Cstage = S1 |
| ELSE IF (BSIHi && REMs >= 4) | |
| IF (Spindle not high && FstWv Pwr High) | Cstage = W |
| ELSE IF (S2Wvs) | Cstage = S2 |
| ELSE | Cstage = S1 |
| ELSE IF (MA >= 3) | |
| IF ((BSIHi && FSPLow) \|\| FstWv Pwr VH \|\| (AftW \|\| AftM)) | Cstage = W |
| ELSE IF (S2Wvs && !BSIHst) | Cstage = S2 |
| ELSE IF (FSPHst) | Cstage = S2 |
| ELSE | Cstage = S1 |
| ELSE IF (AftS2 \|\| AftDS) | |
| IF (MslTVH && AlpPwr Incrs > 200% && BSIHi) | Cstage = W |
| ELSIF (AftDS && EEGPwr Incrs > 50% && Delta Incrs > 10%) | Cstage = S3 |
| ELSE IF (!BSIHi && S2Wvs) | Cstage = S2 |
| ELSE | Cstage = S1 |
| ELSE IF (AftS1 && FstWv Pwr High && AlpPwr Incrs > 20% && | |
| FstWv Pwr Incrs >20% && BSIHi) | Cstage = W |
| ELSE | Cstage = S1 |

Referring in particular to FIG. 19, there is shown a flow chart illustrating a process 1900 describing operation of one embodiment of a sleep stage determination system, such as system 1500 or 1700. For each observational interval or epoch, which may be at a predetermined frequency or at a variable frequency influenced by prior epochs, raw data, sample status and contextual information is collected at step 1905.

Collected data for the current sample is evaluated at step 1910 to determine if it can be categorized as abnormal. Unless the sample is abnormal, the process continues to a full evaluation branch, beginning at step 1945. The sample may be considered to be abnormal if it contains no signal data or only background noise, for example. This may indicate a fault in the sensing unit 1110 or processing unit 1520 or may be due to a disconnection of the sensing unit 1110 from processing unit 1520.

In the event that the current sample is identified as abnormal at step 1910, a set of branch logic rules is evaluated, to diagnose system state and identify sleep stage, if possible. The diagnostic process begins at step 1915 by first determining whether the system has been instructed to stop recording data, for example, if a patient or other person has issued a command through user interface 1660. If so, the current sleep stage is marked as undecided at step 1920. If the system has not been instructed to stop recording, a test is performed by processing unit 1520 at step 1925 to identify whether connecting sensing unit 1110 has been disconnected from processing unit 1520. This may occur intentionally, for example when the patient gets out of bed and leaves the room.

When the sensing unit 1110 is disconnected from the processing unit 1520, the input conductors of the processing unit 1520 may pick up low level background noise. The processing unit 1520 is configured to compare the received low level background noise to a noise level threshold and/or filtering circuit to determine whether the received noise is consistent with a disconnection. Alternatively, the processing unit 1520 may comprise a circuit to sense when the connector 1130 is connected or disconnected from the corresponding connecting part on or associated with processing unit 1520. If the sensing unit 1110 is determined by processing unit 1520 to be disconnected, the current sleep stage is categorized as wake at step 1930. Otherwise it is marked as unscorable at step 1935.

Upon completion of diagnostic tests, a further test is performed to identify if there exist previous undecided epochs at step 1940. If there are none, the current iteration of process 1900 ends. If there exist previous undecided epochs, an evaluation process is invoked at step 1995a to determine previous undecided epochs, before process 1900 is ended.

If the sample status is normal at step 1910, signal preconditioning is performed at step 1945 prior to EEG, EOG and EMG analysis at steps 1950, 1955 and 1960, respectively. Step 1945 is performed by signal conditioning module 1650, whereas digital signal processor 640 and microprocessor 1630 perform steps 1950, 1955 and 1960. After signal analysis at steps 1945 through 1960, staging reasoning is conducted at step 1965 using a process such as that described above with respect to FIG. 18. Upon completion of staging reasoning step 1965, a set of rules is evaluated, beginning at step 1970, to either complete the current iteration of process 1900 or invoke an evaluation module to determine previous undecided epochs.

For each of steps 1950, 1955 and 1960, the respective EEG, EOG and EMG signal analysis is performed in order to determine various characteristics and/or events or parameters indicated by the signals. This analysis may include suitable digital signal processing, including, for example, filtering, sampling Fourier transforms, or threshold comparisons. Such analysis may be carried out in the time domain or frequency domain, as appropriate. For example, the analysis may include analysis of the power spectral density in the frequency domain. Steps 1950, 1955 and 1960 may be performed in the sequence indicated or the order of these steps may be changed or they may be performed simultaneously.

In the signal preconditioning step 1945, the biological signal data is digitized and amplified, if necessary, by signal conditioning unit 1650. Further, digital signal processor 1640 processes the biological signal data to obtain the EEG, EMG and EOG signal data (as described further below), after which the EEG, EMG and EOG signal data are analyzed (as described further below) to provide the processed data referred to above in relation to step 1805.

If the current sleep stage is determined at step 1970 and there are no previous undecided epochs found at step 1985, microprocessor 1630 saves contextual information at step 1990, for example, to data store 1680, and ends the current iteration. If the current sleep stage is determined at step 1970 and there are extant previous undecided epochs at step 1985, an evaluation process is invoked to determine previous undecided epochs at step 1995; upon completion of which the current iteration ends.

If the current sleep stage is not determined at step 1970, the process will save the current epoch with previous undecided epochs at step 1975, for example, to data store 1680. If there exist 6 previous undecided epochs at step 1980 an evaluation process is invoked to determine previous undecided epochs at step 1995; upon completion of which the current iteration ends. If there are fewer than 6 previous undecided epochs at step 1980, the current iteration ends immediately. Using 6 as the upper limit of previous undecided epochs assumes epochs of 30 seconds and that the 3 minute smoothing rule applies, whereby if a K complex or spindle is not seen within 3 minutes of the previous K complex or spindle, the sleep stage defaults to stage one sleep (S1). A predetermined number other than 6 may be used in step 1980 according to alternative configurations, for example where shorter or longer epochs are used.

The evaluation process to determine previous undecided epochs at step 1995, which is performed by undecided epoch categorization module 1688, evaluates prior undecided epochs and last detected epochs, not necessarily in sequential order, to identify patterns that could not otherwise be identified.

Pseudo-code describing the decision process of one example of a determination of previous undecided epochs, such as that performed at steps 1995 and 1995a, is shown below. A glossary of acronyms and abbreviations used in the pseudo-code is provided in Table 3 below.

Determine Previous Undecided Epochs

| | |
|---|---|
| IF (Only one epoch && PUE == R_W, R_S1, R_S2 or R_M) | |
| IF (PUE == R_W) | Cstage = W |
| ELSE IF (PUE == R_S1) | Cstage = S1 |
| ELSE IF (PUE == R_S2) | Cstage = S2 |
| ELSE IF (PUE == R_M) | Cstage = MT |
| ELSE IF (NDE == W) | |
| LOOP the Previous Undecided Epochs List | |
| IF (PUE == REM_S1) | Cstage = S1 |
| ELSE IF (PUE == R_S2) | Cstage = S2 |
| ELSE IF (PUE == R_M) | Cstage = MT |
| ELSE | Cstage = W |
| ELSE IF (NDE == REM) | |
| LOOP the Previous Undecided Epochs List | |
| IF (PUE == R_W, R_M, W or MT) | |
| IF (LDE == W or MT) | Cstage = W |
| ELSE | Cstage = MT |
| ELSE | Cstage = REM |
| ELSE IF (NDE == MT) | |
| LOOP the Previous Undecided Epochs List | |
| IF (PUE == R_S1) | Cstage = S1 |
| ELSE IF (PUE == R_S2) | Cstage = S2 |
| ELSE | Cstage = W |
| ELSE | |
| LOOP the Previous Undecided Epochs List | |
| IF (LDE == S2) | Cstage = S2 |
| ELSE IF (LDE == REM) | Cstage = REM |
| ELSE IF (PUE == R_W or R_M) | Cstage = MT |
| ELSE IF (PUE == R_S1) | Cstage = S1 |
| ELSE IF (PUE == R_S2) | Cstage = S2 |
| ELSE | Cstage = NDE |

Upon completion of the determination of previous undecided epochs at steps 1995 and 1995a, contextual information for the relevant epochs is saved in a data store, such as data store 1680.

Processing and analysis of the biological signal data to obtain the EEG, EMG and EOG data may be performed as described below. The EEG signals are derived from channels associated with electrode pairs E1-E3 and E2-E3. The EEG signals are obtained by filtering the biological signal data with a band pass filter to obtain frequencies between 0.5 and 30 Hz.

Each epoch of 30 seconds is divided into 10 equal segments and each 3-second data segment is subject to a Fast Fourier Transform (FFT) to obtain the power spectral density for each EEG sub activity. Table 1 below shows the frequency bands for a number of recognized EEG sub activities.

TABLE 1
Frequency ranges of EEG sub activities

| Frequency | Alpha | Beta1 | Beta2 | Delta | Sigma | Theta |
|---|---|---|---|---|---|---|
| Low | 7.5 | 16.0 | 20.0 | 1.0 | 12.0 | 3.0 |
| High | 9.5 | 20.0 | 28.0 | 2.5 | 15.0 | 7.0 |

Thus, for example, for each 3 second data segment, digital signal processor 1640 calculates the power of the EEG signals in the Alpha band between 7.5 and 9.5 Hz, and so on for each of the other EEG sub activities. In addition to the power spectral analysis of each 3-second data segment, several parameters are calculated by DSP 640, including a Beta/Sigma index (BSI), an Alpha/Theta index (ATI) and the frontal spindle (FSP). The BSI is the ratio between the power of the Beta2 band and that of the Sigma band. The ATI is the ratio between the power of the Alpha band and that of the Theta band. The FSP is the calculated spectral power of the sigma band.

Further features are extracted from the EEG signal data that involve determination of the total duration for which the signal data is within a certain amplitude range for each epoch. Specifically, Delta, Spindle and K-complex activities are extracted from the EEG signals, based on the frequency and amplitude parameters listed in Table 2 below.

TABLE 2
Parameters for detection of delta, spindle, K-complex

| Parameters | Delta | Spindle | K-complex |
|---|---|---|---|
| Filter frequency (Hz) | 0.5-3.5 | 9.0-15.0 | 3.0-7.0 |
| Amplitude range (μV) | 35-150 | 15-60 | 30-70 |

The parameters listed in Table 2 are used to calculate the proportion of the epoch for which the EEG signal data is within the amplitude range indicated for the listed delta, spindle and K-complex frequencies. For example, if greater than 50% of the epoch has signals in the amplitude range of 35 to 150 μV for delta frequencies between 0.5 and 3.5 Hz, then this information may be used to determine that the epoch should be decided as belonging to deep sleep stage S4. If greater than 20% of the epoch is within the amplitude range for the delta frequencies, then the epoch may be decided as belonging to deep sleep stage S3. Deep sleep stages S3 and S4 may be collectively scored as “deep sleep” if the amplitude range for the delta frequencies exceeds a minimum threshold. Such a minimum threshold is configurable, but may be between 2% and 20%, for example.

The EMG analysis of the biological signal data is performed based on the signals received from channel E1-E2 and band-pass filtered between 20 and 40 Hz. The EMG analysis involves calculation of tonic and phasic activities of muscles in the forehead and vicinity. Tonic activity is associated with a relaxed, restful state and is referred to also as muscle tone or tonus. Tonic activity is used to detect changes in muscle tone for REM and NREM sleep. Phasic activity is associated with sudden increases in muscle activity and is used for the detection of movement arousal.

To avoid EMG bursts that may be associated with the phasic activity, which can be a result of a muscle twitch or movement, obscuring the tonic activity, the tonic activity is calculated using the inter-quartile range method, instead of integrating the rectified EMG data of the whole epoch. The inter-quartile range is calculated as the difference between the 75th percentile of sample amplitudes (often called Q3) and the 25th percentile (called Q1). The inter-quartile range is also sometimes called the H-spread. Period analysis is used to detect peaks in the EMG signal data and the amplitudes of the detected peaks are sorted and the muscle tone is then calculated as the difference between Q3 and Q1. The calculated EMG tonus is used as a threshold to detect EMG burst phasic activities.

EMG burst detection is performed in a similar manner to the EEG feature extraction described above, but with a frequency range of 10 to 40 Hz. If any EMG bursts are detected in an epoch, the 3-second data segments including the detected EMG bursts are eliminated from consideration and the rest of the data segments in the epoch are used to again calculate the EMG tonus. Additionally, DSP 1640 determines the average amplitude of the EMG tonus, as this may be relevant to determination of which sleep stage the epoch should be assigned to. For example, if the average amplitude of the EMG tonus is low, then this indicates a REM sleep stage.

For the EOG analysis, left and right EOG signal data are extracted from the biological signal data via channels E1-E3 and E2-E3 band-pass filtered between 0.5 and 30 Hz. The EOG signal data are analyzed by DSP 1640 for eye activities, including eye blink, rapid eye movement and slow eye movement. The EOG signal data is analyzed with respect to an amplitude threshold, frequency range (peak to peak intervals), rising slope and falling slope of the EOG signal wave form to detect eye movements. Wave forms in the EOG signal data corresponding to eye movements are detected separately between the left and right EOG channels. Only those eye movements that are negatively correlated from left and right EOG channels are considered as true.

Some high amplitude (up to several hundred micro volts), low frequency (0.1 to 1.0 Hz) waveforms may be detected in frontal channels. The waveforms may be, for example, EEG delta waves, eye movement activities, or signal noise generated by body movements or other sources. With limited information available from the frontal channels, it may be difficult to identify the source of such waveforms, which in turn may result in difficulties in determining the sleep stage. For example, false detection of eye movements or delta activities may be caused by noise associated with body movements. As a result, it may be difficult to distinguish Wake from stage 1, stage 2 from deep sleep, REM from stage 1, and Wake from deep sleep when alpha intrusion occurs in deep sleep.

In one embodiment, one or more body movement sensors may be used for transmitting signals to processing unit 1520 or pre-processing unit 1720 over a body movement channel. Information or signals received over the body movement channel can be helpful for sleep staging with frontal channels by assisting to identify the source of the high amplitude slow waveforms (HASWs). For example, when body movement is detected (with the body movement channel), the HASWs can be considered to be noise and the epoch can be scored as either Wake or MT (movement time). Further, if the epoch immediately follows or precedes a Wake stage, or if there is dominant alpha activity found in the current epoch, then the epoch can be scored as Wake. Otherwise, the epoch is scored as MT.

When no body movement is detected, the HASWs can be regarded as eye movements or delta activities and the stage of the epoch can therefore be narrowed down to Wake, REM or S1 if the background activities are dominated by fast activities (alpha, beta activities); or the epoch is scored as S2 or delta sleep if the dominant activities are slow waveforms (delta activities). The exact stage is determined by other information, for example such as contextual information, EEG power spectra, and percentage of duration of detected delta activities over the epoch.

The one or more body movement sensors for providing signal data over the body movement channel are positioned on, or relative to, a part of the body away from the head. Such sensors may sense movement by use of one or more accelerometers affixed to the body or they may sense movement by detection of EMG signals derived from an EMG sensor, for example. Alternatively, the body movement sensors may be located away from the body and may employ or promote movement detection techniques, such as are known in the art, including, but not limited to, optical imaging. An example of a suitable accelerometer for use in the one or more body movement sensors is an accelerometer made by FreeScale Semiconductor, Inc. of Austin, Tex. under the MMA7260Q product series.

The body movement sensors may be coupled to processing unit 1520 or pre-processing unit 1720 directly or via sensing unit 1110. Communication of the signal data obtained by the body movement sensors to processing unit 1520 or pre-processing unit 1720 may be by way of a wired or wireless connection. If an EMG signal detection component is used in the one or more body movement sensors, it may be coupled with a wireless transmitter, as described in U.S. patent application Ser. No. 11/130,221.

Provision of the described means for detecting body movement enables the identification of HASWs in each epoch being considered, which in turn assists in determining the correct sleep stage of the subject.

Further embodiments relate to use of the described mask assemblies and/or sensing unit comprising the frontal electrode array, in combination with a flow sensor, blood oximeter and processor, to determine an apnea-hypopnea index (AHI) of the wearer. Such embodiments make use of the described method of sleep stage determination to determine the sleep stage of the wearer of the frontal electrode array, or at least to determine whether the wearer is awake or asleep.

At a minimum, AHI calculation according to the described embodiments involves determining the sleep state of the patient using the frontal electrode array, sensing respiratory air flow and sensing the blood oxygen saturation level of the patient. Based on the sleep state and respiratory flow, an apnea event can be determined and, based on the sleep state and respiratory flow and at least one of the blood oxygen saturation and the sleep arousal state of the person, a hypopnea event can be determined. Depending on whether the embodiments are directed to diagnosis of a sleep-related breathing disorder, such as OSA, or determining the efficacy of treatment of the disorder, the embodiments may include supplying breathing gas to the wearer at a positive airway pressure. For diagnosis purposes, no such breathing gas is supplied or it is supplied at a sub-therapeutic level, but for determining the efficacy of treatment, the breathing gas must be supplied at a therapeutic level.

The AHI is the sum of apneas and hypopneas per hour of sleep and is an indication of the severity of the sleep-related breathing disorder. If the AHI is greater than an upper threshold value such as 15, or if the AHI is greater than a lower threshold value such as 5 and the patient suffers from excessive daytime sleepiness, this indicates a diagnosis of OSA. The treatment of choice for OSA is commonly a CPAP therapeutic appliance.

According to the American Academy of Sleep Medicine (AASM), an apnea event is defined as a 50% or greater drop of flow lasting at least 10 seconds that occurs during sleep. Hypopnea is defined clinically as a 30% or greater drop in flow lasting at least 10 seconds associated with a minimum 4% oxygen desaturation that occurs during sleep. An alternate research definition of hypopnea is defined as a 30% or greater drop in flow lasting at least 10 seconds associated with either a minimum 4% oxygen desaturation or an arousal that occurs during sleep. Other criteria for determining the occurrence of apnea and hypopnea events may be used, based on different clinically acceptable respiratory flow, oxygen desaturation levels and occurrence of arousals. The AHI is calculated as the sum of all apnea and hypopnea events divided by the number of hours of sleep time.

Through the use of a frontal electrode array (as described herein) located on the PAP mask or headgear, the EEG, EMG and EOG from the forehead is analyzed to determine the sleep state of the patient. This frontal EEG can also be analyzed to determine the presence of arousals. An EEG arousal is defined according to the MSM as an abrupt shift in EEG frequency, which may include theta, alpha, and/or frequencies greater than 16 Hz but not spindles. Oxygen desaturation events can be determined from an oximeter measuring the blood oxygen saturation with a transducer located on a finger, ear or forehead. Flow limitations can be determined from measurements of airflow via a pneumotachograph located in the CPAP device, or by sensing the pressure at the nasal or naso-oral interface, or by a thermistor located in the nasal or naso-oral interface.

The sleep state, coupled with the measurement of respiratory gas flow, can be used to determine apnea events. The sleep state, coupled with flow, arousals and desaturations, can be used to determine hypopnea events. From these events, and measurements of the total time asleep derived from the bioelectric signals received at the frontal electrodes, the AHI can be calculated. The AHI can be used as a measure of the efficacy of therapy.

The identification of apneas and hypopneas can also be used to adjust the level of therapeutic pressure applied by the CPAP device. Currently, Auto-adjusting PAP devices are available which ramp up the pressure based on breathing events. An improvement to this method is to adjust the pressure based on the detection of apneas and hypopneas—breathing events that occur while the patient is asleep. Another improvement is to increase pressure further during REM sleep, which is typically associated with more apnea events.

The calculation of AHI based on frontal EEG and other parameters can also be used to diagnose sleep disorders. The frontal electrodes with mask and headgear are applied along with the oximetry and flow sensors. The PAP can be set to provide a low, non-therapeutic level of pressure, or turned off to generate no regulated level of pressure. The resulting AHI can then be used to aid in the diagnosis of OSA.

In other embodiments, the PAP device can be set to provide a non-therapeutic pressure or no regulated pressure for the first portion of the designated period for the sleep study, and to supply therapeutic pressure during the second portion of the designated period. This is known as a “split-night” study. For embodiments in which the CPAP device is used without pressure regulation, an anti-asphyxia valve is provided in the air supply interface, the supply tube or the PAP device.

For a split-night study, the duration of the first portion of the designated period may be determined by at least one of the following rules: a minimum or maximum duration; a minimum or maximum number of apnea or hypopnea events; a minimum or maximum number of apnea or hypopnea events in a given sleep stage; a minimum or maximum time spent in a given sleep stage or combination of stages; and a minimum or maximum time spent in a given body position. The second portion of the designated period may be used to determine the correct level of therapeutic pressure. For example, the pressure may be increased until the measured respiratory events in specific sleep stages and body positions are reduced to an acceptable level.

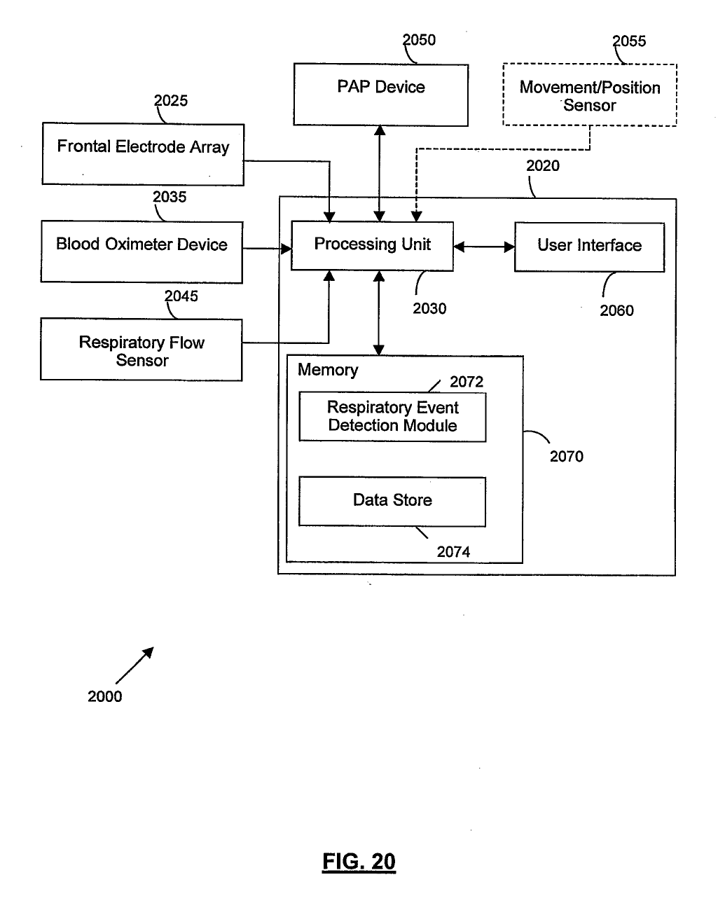

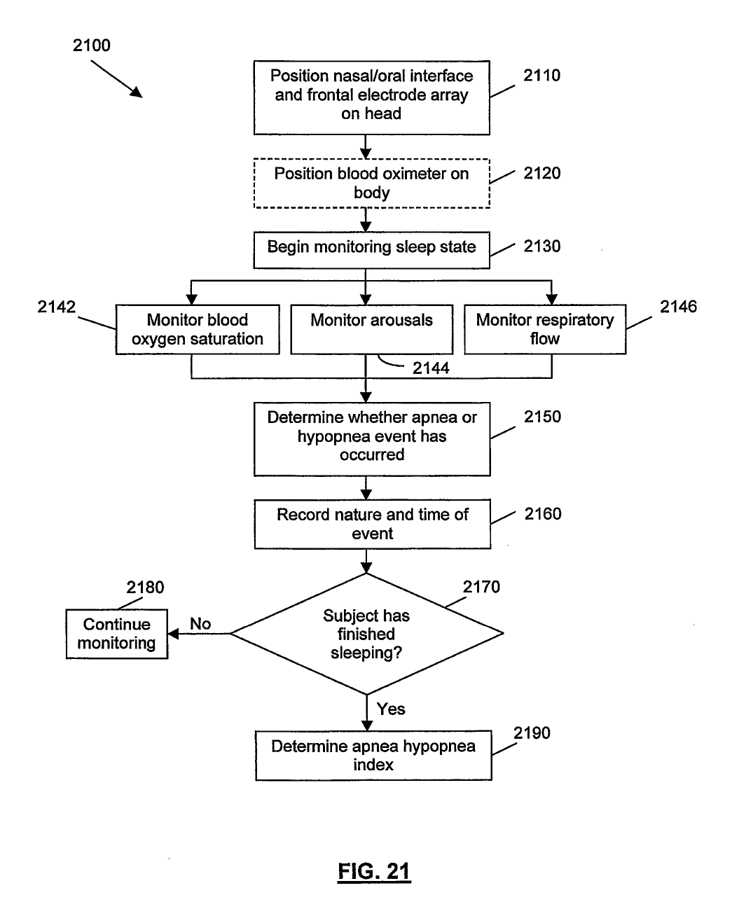

Some embodiments relate to systems and devices for determining the occurrence of respiratory events. Examples of such systems and devices are described above in relation to FIGS. 1 to 17. A further example of such systems and devices is described below in relation to FIG. 20. Such systems and devices may be used to calculate the AHI for wearer 18 based on detected apnea and hypopnea events and, based on the AHI and whether breathing gas is supplied to wearer 18, to also determine results of diagnostic relevance in relation to a sleep-related disorder, such as OSA, or to determine an indication of the therapeutic efficacy of the supplied breathing gas.

FIG. 20 is an illustrative block diagram of some embodiments of a system 2000 for use in determining the occurrence of respiratory events of wearer 18. System 2000 comprises a computer system 2020 and, coupled thereto, a frontal electrode array 2025, a blood oximeter device 2035, a respiratory flow sensor 2045 and a PAP device 2050. Optionally, a movement/position sensor 2055 is also coupled to computer system 2020.

A nasal or naso-oral interface, such as is described above in relation to FIGS. 1 to 10, may be comprised in PAP device 2050 and used to supply the breathing gas. The frontal electrode array 2025 is employed as described above to gather bio-electric signal data from which the wearer's sleep stage can be determined. The blood oximeter device 2035 can be located on sensing unit 1110, forehead plate 214, 714 or 814, or on another location on the head or on a body part away from the head such as a finger, and is used to determine the blood oxygen saturation of wearer 18. The respiratory flow measurement device 2045, which may be a flow or pressure sensor coupled to a breathing gas conduit, is used to measure respiratory flow of wearer 18.

Computer system 2020 comprises a processing unit 2030, analogous to processing unit 1520, 414, 514 or monitoring unit 16, that is coupled to the frontal electrode array 2025, the blood oximeter device 2035, the respiratory flow measurement device 2045 and the PAP device 2050 and is configured to determine the occurrence of an apnea event and a hypopnea event based on the signals it receives from those devices.

The processing unit 2030 is further configured to determine the time in which the wearer 18 is in a sleep state that is not awake over a period designated for sleep by the wearer 18. The processing unit 2030 is configured to determine the AHI of the wearer 18 for the sleep period based on the determined apnea and hypopnea events and the total time period (or a portion thereof in which the wearer 18 was asleep. This calculated AHI can then be used by the processing unit to determine a likely indication of the existence of a sleep related disorder in wearer 18 or to determine the efficacy of PAP treatment provided to the wearer 18.

Determination of respiratory events and calculation of relevant indicators in relation to such events, such as AHI, is performed by processing unit 2030 executing a respiratory event detection module 2072 stored as executable program instructions in memory 2070. Memory 2070 also comprises a data store 2074 accessible to processing unit 2030 as necessary for storing and retrieving data extracted from the measurement signals from frontal electrode array 2025, blood oximeter device 2035, respiratory flow sensor 2045 and, optionally, movement/position sensor 2055. Such data is processed by processing unit 2030 executing respiratory event detection module 2072 to detect the occurrence of respiratory events and for subsequent determination of indicators, such as AHI or a respiratory distress index (RDI), and to compare such indicators against established threshold values to provide a diagnostic indication or an indication of the efficacy of therapy. Memory 2070 may also comprise the modules of memory 1670 described above in relation to FIG. 16 for enabling computer system 2020 to determine the sleep stage of the wearer 18 as part of determining the occurrence of respiratory events.

In addition to apnea and hypopnea respiratory events, other respiratory events that may be detected using the described embodiments include upper airway resistance (UAR) and a respiratory effort related arousal (RERA). An UAR event corresponds to a flattening of the respiratory flow curve during sleep and is an early indicator of an apnea event,. A RERA may be defined as a sequence of breaths that is characterized by increasing respiratory effort, leading to an arousal from sleep that does not meet the criteria for an apnea or hypopnea. The sequence of breaths must last at least ten (10) seconds and show a pattern of progressively more negative esophageal pressure, terminated by a sudden change in pressure to a less negative level, together with an arousal. Each RERA event can be tracked by respiratory event detection module 2072 and used with detected apneas and hypopneas to calculate the RDI for a period of time in which wearer 18 is asleep.

Where the AHI or RDI is above a clinically determined level, this may be used by the processing unit to provide an indication of treatment efficacy or the existence of a sleep related disorder. For example, if positive airway pressure is being supplied to wearer 18 according to a prescribed PAP treatment plan, but the AHI or RDI is sufficiently high to indicate that a number of apnea and/or hypopnea events occurred during the sleep period despite the treatment, this can be considered to be an indication that the prescribed PAP treatment is not as effective as intended.

Referring also to FIG. 21, a method 2100 of determining the occurrence of respiratory events is described in further detail. The method 2100 begins at step 2110, in which the nasal or naso-oral interface and frontal electrode array 2025 are positioned on the wearer's head 18. Additionally, if the blood oximeter device 2035 is not integrated with the forehead member on which the frontal electrode array 2025 is located, then the blood oximeter device 2035 is positioned on the wearer's body at step 2120, for example by attachment to the earlobe or a finger.

At step 2130, the system 2000 begins monitoring the sleep state of the wearer 18 by causing processing unit 2030 to execute respiratory event detection module 2072. While monitoring the sleep state, the system 2000 also monitors the blood oxygen saturation level at step 2142, monitors arousals at step 2144 and monitors respiratory flow at step 2146.

At step 2150, the processing unit 2030 determines whether an apnea or hypopnea event has occurred based on the monitored sleep state, blood oxygen saturation (and/or arousals) and respiratory flow. If an apnea or hypopnea (or other respiratory) event is determined to have occurred at step 2150, the nature (i.e. whether apnea, hypopnea, RERA or other) and time of the event is recorded at step 2160, together with any other diagnostically or analytically relevant data, such as may be relevant to a split-night study.

At step 2170, the processing unit 2030 checks whether the subject (i.e. the wearer 18) has finished sleeping. This may be determined according to the planned sleep period of the wearer 18 or based on the wearer being awake for an extended period following an extended period of being asleep. If the system determines that the subject has finished sleeping, then at step 2190, the processing unit 2030 determines the apnea-hypopnea index or RDI based on the determined apnea, hypopnea and RERA events and the time that the subject was asleep during the designated period. Step 2190 may also comprise determining an AHI or RDI for only part of the designated period or for multiple sub-portions of the designated period. As long as the processing unit 2030 determines that the subject has not finished sleeping at step 2170, the processing unit 2030 continues to monitor for respiratory events at step 2180 (i.e. by repeating steps 2130 to 2160).

It may also be desirable for the processing unit 2030 to calculate the AHI or RDI at periodic intervals throughout the night based on the current amount of sleep time. This would be useful in a monitoring situation, during PAP titration, or to determine the completion of the diagnostic portion of a split night study. It is also of interest to calculate the AHI in specific sleep stages (eg: REM) and in specific body positions (eg: supine). The movement/position sensor 2055 may be used to detect the body position of wearer 18.

It should be understood that features shown and described in relation to each of the embodiments may be used in combination or substitution with any features of the other described embodiments, where such a combination or substitution would not result in an unworkable arrangement or configuration. Accordingly, the present invention is contemplated to encompass all such combinations or substitutions resulting in operative embodiments.

While the above description provides examples of embodiments, it will be appreciated that some features and/or functions of the described embodiments are susceptible to modification and change without departing from the spirit and principles of operation of the described embodiments. For instance, the described embodiments are applicable to other types of gas delivery devices such as variable positive air pressure devices bi-level positive air pressure devices, auto-adjustable air pressure devices, demand positive pressure devices and other variations of such devices. The described embodiments can also be used in other instances where an individual wears a mask as well as sensors for gathering physiological data, such as in critical care units. In addition, it should be understood that the particular air and sensor interfaces shown and described herein are shown as examples only and that at least some of the described embodiments may be applicable to other mask designs. Accordingly, what has been described and shown in the drawings is intended to be illustrative of the invention and the described embodiments, rather than being a limiting and/or exclusive definition.

TABLE 3
Glossary of terms, acronyms and abbreviations

| Acronym or | |
|---|---|
| Abbreviation | Description and Definition |
| AftDS | The immediate previous epoch is scored as Deep Sleep (S3 or S4) |
| AftM | The immediate previous epoch is scored as MT |
| AftR | The immediate previous epoch is scored as REM |
| AftRLike | The immediate previous epoch is scored as R_M, R_W, R_S1 or R_S2 |
| AftR_M | An epoch which is scored as Movement Time for which the immediate |
| | previous epoch is scored as REM |
| AftR_W | An epoch which is scored as Wake for which the immediate previous |
| | epoch is scored as REM |
| AftS1 | The immediate previous epoch is scored as S1 |
| AftS2 | The immediate previous epoch is scored as S2 |
| AftW | The immediate previous epoch is scored as Wake |
| Alp | EEG alpha sub band: 7.5˜9.5 Hz |
| AlpEEG | Alpha type EEG: Peak found in the alpha sub band in Wake epochs. |
| AlpPk | Peak found in the alpha sub band on EEG power spectra |
| AlpPwr | EEG power of spectra of alpha sub band |
| AlpPwrLow | AlpPwr is low when it is < the average AlpPwr of previous S1 epochs |
| AlpPwrHi | AlpPwr is high when it is: |
| | >2 times of the average AlpPwr of previous S1, OR |
| | >Average AlpPwr of wake epochs without eye movements |
| ASI | Ratio of Alpha and Spindle band EEG power spectra |
| ATI | Alpha Theta Index: Ratio of Alpha and Theta band EEG power spectra |
| ATILow | Alpha Theta Index Low: when the ratio of alpha and theta sub bands |
| | power of spectra is <0.4. |
| Bta1 | EEG beta1 sub band: 16˜20 Hz |
| Bta1Pwr | EEG power of spectra of Bta1 sub band |
| Bta2 | EEG beta2 sub band: 20˜28 Hz |
| BtaEEG | Beta type EEG: Peak found in the beta sub band in Wake epochs. |
| Bta2Pwr | EEG power of spectra of Bta2 sub band |
| BtaPk | Peak found in the Bta1 sub band on EEG power spectra |
| BSI | Ratio of Bta2 and Spindle band EEG power spectra |
| BSIHi | Current BSI level is high if it is not BSIHst, AND: |
| | >50% of its average over previous S1, REM and Wake epochs, OR |
| | >2 times its average over previous S2 epochs; |
| | >1.5 |
| BSIHst | Current BSI level is highest if it is: |
| | >its average over previous S1, REM epochs or 80% of wake epoch |
| | average, OR |
| | >2.0 AND >50% of its average over previous S1 epochs, OR |
| | >3.0 |
| BSILow | Current BSI level is low if it is not BSIHst, not BSIHi, not BSILwst, AND: |
| | <its average over previous SD epochs, OR |
| | <1.2 times its average over previous S2 epochs, OR |
| | <0.5 |
| BSILwst | Current BSI level is lowest if it is not BSIHst, not BSIHi, AND: |
| | <<Of its average over previous S2 epochs, OR |
| | <20% of its average over previous REM epochs, OR |
| | <0.1 |
| BSI VH | BSI very High if: |
| | BSIHst or |
| | BSIHi and BSI increased more than 50%. |
| Cstage | Stage of current epoch (being analyzed) |
| Decrs | Decreased compared to last epoch (e.g. Alpha Incrs >0.2 = alpha |
| | decreased more than 20% than last epoch) |
| Del | EEG delta sub band: 1˜2.5 Hz |
| DelPwr VH | EEG power of spectra of delta sub band is very high, when it is: |
| | >5 × 10⁷, μV² AND |
| | >2 times its value in previous S2, AND |
| | ATI <0.4. |
| Delta | Duration of detected delta waves (in seconds). |
| EEGPwr | EEG power of spectra of the sub band ranging from 1˜28 Hz. |
| EEGPwr Low | EEGPwr is low when it is: |
| | <10% of the average of previous wake epochs with eye movements; |
| | OR |
| | <20% of the average of wakes epochs without eye movements. |
| EEGPwr VH | EEGPwr very high when it is: |
| | >the average of previous wake epochs with eye movements; OR |
| | >3 times the average of previous wake epochs without eye movements. |
| FSP | Frontal spindle: 10.5˜14 Hz |
| FSPHi | Current FSPPwr level is high if it is not FSPLwst, not FSPLow and not |
| | FSPHst. |
| FSPHst | Current FSPPwr level is low if it is not FSPLwst and not FSPLow, AND: |
| | >its average over previous SD epochs, OR |
| | >80% of its average over previous S2 epochs, OR |
| | >3 times its average over previous S1 epochs, OR |
| | >4 times its average over previous wake epochs. |
| FSPLow | Current FSPPwr level is low if it is not FSPLwst, AND: |
| | <its average over previous S1 epochs, OR |
| | <1.2 times its average over previous REM epochs, OR |
| | <50% of its average over previous S2 epochs. |
| FSPLwst | Current FSPPwr level is lowest if it is: |
| | <its average over previous REM, wake epochs, or 80% of S1 epochs; |
| | OR |
| | <30% of its average over previous S2 epochs. |
| FSPPwr | Value of EEG power spectra of frontal spindle (ranging from 10.5˜14 Hz) |
| FstWv | EEG power spectra of fast waves (ranging from 8 to 30 Hz). |
| FstWv Pwr | FstWv Pwr is high when it is: |
| High | >its average over previous S1 epochs, AND |
| | >7.5 × 10⁶ μV². |
| FstWv Pwr | FstWv is low when it is < its average value of previous S1 epochs. |
| Low | |
| FstWv Pwr | FstWv is very high when it is > its average value of previous Wake |
| VH | epochs. |
| FEMs | Number of Fast Eye Movements: REMs + eye blinks; |
| HBSI | The number of segments (out of total 10 for each epoch) for which BSI |
| | is BSIHi. Each 30 second epoch is divided evenly into ten 3 second |
| | segments. The EEG power spectra of each is analyzed independently |
| | and categorized. |
| Incrs | Increased compared to last epoch (e.g. Alpha Incrs >0.2 = alpha |
| | increased more than 20% than last epoch) |
| LDE | Last determined epoch: the epoch for which a sleep stage was |
| | determined and which is immediately BEFORE current previous |
| | undecided epoch. |
| LFSP | The number of segments (out of total 10 for each epoch) for which FSP |
| | is FSPLow |
| MA | Duration of detected movement arousal (in seconds) |
| MslTLow | Muscle tone level is Low, if it is: |
| | <its average over previous S1, S2 and SD epochs; OR |
| | <1.2 times its average over previous REM epochs. |
| MslTVH | Muscle tone level is very high, if it is: |
| | >2 times its average value of previous S1, S2, SD and REM epochs. |
| MT | Movement Time sleep stage |
| NDE | Next determined epoch: the epoch for which a sleep stage was |
| | determined and which is immediately AFTER current previous |
| | undecided epoch. |
| Noisy | The signals are noisy: more than 50% of the epoch in which signal |
| | amplitude is higher than 200 μV for EEG, 500 μV for EMG and 300 μV |
| | for EOGs. |
| PUE | Previous undecided epochs |
| REM | Sleep stage REM |
| REMBgrd | REM background activities when: |
| | MslTLow, AND |
| | AftR, AND |
| | REMs >0, AND |
| | !BSILwst, AND |
| | !AlpPk, AND |
| | !FstWv Pwr VH, AND |
| | FSPLow |
| REMs | Number of detected rapid eye movement(s) |
| R_M | REM or MT |
| R_S1 | REM or S1 |
| R_S2 | REM or S2 |
| R_W | REM or Wake |
| S1 | Sleep stage 1 |
| S2 | Sleep stage 2 |
| SD | Delta (deep) sleep stage (S3 or S4) |
| S2Wvs | Spindle, K-Complex found in the epoch |
| Spindle not | Frontal spindle activities are not high when: the duration of detected |
| high | spindles <10% of the epoch length AND FSPLow |
| SpnPk | Peak found in the FSP sub band on EEG power spectra |
| Tht | EEG theta sub band: 3˜7 Hz |
| Tht Pwr Low | Theta sub band power of EEG spectra is low when it is: |
| | <2.0 times its lowest value. |
| W | Sleep stage Wake |
| Wakening | Wakening activities: when MsITVH, BSIHi or BSIHst, and AlpPwr |
| | increased more than 200%. |
| && | Logic AND |
| \|\| | Logic OR |
| ! | Logic NOT |

1. A system for determining the occurrence of respiratory events, comprising:
a plurality of electrodes positionable at frontal locations on a person's forehead to sense bioelectric signals;
a measurement device for measuring respiratory flow of the person;
a blood oximeter device for measuring a blood oxygen saturation of the person; and
a processing unit coupled to the plurality of electrodes, the flow measurement device and the blood oximeter device, wherein the processing unit is configured to determine a sleep state of the person based on the bioelectric signals, to determine the occurrence of an apnea event based on the sleep state and the respiratory flow and to determine the occurrence of a hypopnea event based on the respiratory flow, the sleep state of the person and the blood oxygen saturation.

2. The system of claim 1, wherein the sleep state is one of awake and asleep.

3. The system of claim 1, wherein the sleep state is one of wake, sleep stage one, sleep stage two, deep sleep, REM sleep and movement time.

4. The system of claim 1, wherein the plurality of electrodes are located on a forehead member for positioning on the forehead.

5. The system of claim 4, wherein the plurality of electrodes comprises a first electrode located on a projecting portion of the forehead member for positioning adjacent a nasion area of the head, a second electrode located on the forehead member for positioning over a first lateral forehead area and a third electrode located on the forehead member for positioning over a second lateral forehead area opposite the first lateral forehead area.

6. The system of claim 5, wherein conductors are formed on the forehead member for electrically coupling the first, second and third electrodes to an output connector.

7. The system of claim 5, further comprising a fourth electrode located on the forehead member intermediate the second and third electrodes for positioning over a central forehead area.

8. The system of claim 5, wherein the second and third electrodes are located on the forehead member for positioning higher on the forehead than Fp1 and Fp2 electrode positions.

9. The system of claim 5, wherein the second and third electrodes are located on the forehead member for positioining laterally beyond respective Fp1 and Fp2 electrode positions.

10. The system of claim 4, wherein the forehead member is flexible to accommodate varying forehead shapes.

11. The system of claim 10, wherein the forehead member comprises a flexible plastic substrate.

12. The system of claim 10, wherein the forehead member comprises a woven material.

13. The system of claim 4, wherein the first, second and third electrodes are removeably attachable to the forehead member.

14. The system of claim 1, wherein the processing unit is configured to determine the sleep state based on a plurality of rules applied in relation to the bioelectric signals.

15. The system of claim 14, further comprising a signal conditioning unit coupled to the processing unit for receiving detected electrical potentials from the plurality of electrodes, conditioning the electrical potentials to generate the biological signals and providing the biological signals to the processing unit.

16. The system of claim 14, wherein the plurality of rules are empirically derived based on correlation of physiological conditions with particular biological signals or signal patterns.

17. The system of claim 1, further comprising one or more sensors coupled to the processor and located away from the head for detecting movement of a body of the patient.

18. The system of claim 32, wherein the one or more sensors comprise one or more accelerometers.

19. The system of claim 32, wherein the one or more sensors comprise one or more electromyographic sensors.

20. The system of claim 4, wherein the blood oximeter device is located on the forehead member.

21. The system of claim 1, wherein the blood oximeter device is located on a finger or ear of the person.

22. The system of claim 1, further comprising an air supply interface for supplying positive airway pressure to an airway of the person, wherein the processing unit is configured to control the supply of positive airway pressure.

23. The system of claim 37, wherein the air supply interface comprises a nasal or naso-oral interface.

24. The system of claim 22, wherein the processing unit is further configured to determine a time duration within a designated period that the person is in a sleep state in which the person is asleep.

25. The system of claim 24, wherein the processing unit is further configured to determine an apnea-hypopnea index (AHI) based on the time duration and the number of occurrences of apnea and hypopnea events during at least part of the time duration.

26. The system of claim 25, wherein the processing unit is further configured to determine a therapeutic efficacy of the supplied positive airway pressure based on the AHI.

27. The system of claim 1, wherein the processing unit is further configured to determine a time duration that the person is in a sleep state in which the person is asleep.

28. The system of claim 27, wherein the processing unit is further configured to determine an apnea-hypopnea index (AHI) based on the time duration and the number of occurrences of apnea and hypopnea events during at least part of the time duration.

29. The system of claim 28, wherein the processing unit is further configured to determine whether the person has a sleep-related disorder based on the AHI.

30. The system of claim 27, wherein the processing unit is further configured to determine the occurrence of a respiratory effort related arousal (RERA) event based on the bioelectric signals and the measured respiratory flow.

31. The system of claim 30, wherein the processing unit is further configured to determine a respiratory distress index (RDI) based on the number of occurrences of apnea, hypopnea and RERA events during at least part of the time duration.

32. The system of claim 24, wherein the processing unit is further configured to monitor the occurrence of respiratory events during a first part of the designated period without supplying positive airway pressure to the airway and to monitor the occurrence of respiratory events during a second part of the designated period while supplying positive airway pressure to the airway.

33. The system of claim 32, wherein the processing unit is further configured to end the first part of the designated period and begin the second part of the designated period in response to determining that one or more criteria are satisfied in relation to the occurrence of respiratory events during the first part of the designated period.

34. The system of claim 1, wherein the processing unit is further configured to determine the occurrence of an arousal event based on the bioelectric signals and further configured to determine the occurrence of the hypopnea event based on the respiratory flow, the sleep state of the person and at least one of the arousal event and the blood oxygen saturation.

35. A method of determining the occurrence of respiratory events, comprising:
positioning a plurality of electrodes at frontal locations on a person's forehead;
sensing bioelectric signals using the plurality of electrodes;
measuring respiratory flow of the person;
measuring a blood oxygen saturation of the person;
determining a sleep state of the person based on the bioelectric signals;
determining occurrence of an apnea event based on the sleep state and the respiratory flow; and
determining occurrence of a hypopnea event based on the respiratory flow, the blood oxygen saturation and the sleep state of the person.

36. The method of claim 35, further comprising determining a time duration within a designated period that the person is in a sleep state in which the person is asleep.

37. The method of claim 36, further comprising determining an apnea-hypopnea index (AHI) based on the time duration and the number of occurrences of apnea and hypopnea events during the time duration.

38. The method of claim 37, further comprising determining whether the person has a sleep-related disorder based on the AHI.

39. The method of claim 37, further comprising supplying positive airway pressure to an airway of the person during at least part of the time duration.

40. The method of claim 39, further comprising determining a therapeutic efficacy of the supplied airway pressure based on the AHI.

41. The method of claim 36, further comprising determining occurrence of a respiratory effort related arousal (RERA) event based on the bioelectric signals and the measure respiratory flow.

42. The method of claim 41, further comprising determining a respiratory distress index (RDI) based on the number of occurrences of apnea, hypopnea and RERA events during at least part of the time duration.

43. The method of claim 36, further comprising monitoring the occurrence of respiratory events during a first part of the designated period without supplying positive airway pressure to an airway of the person and monitoring the occurrence of respiratory events during a second part of the designated period while supplying positive airway pressure to the airway.

44. The method of claim 43, further comprising ending the first part of the designated period and beginning the second part of the designated period in response to satisfaction of one or more criteria in relation to at least one of: the occurrence of respiratory events during the first part of the designated period; time spent in one or more sleep states; time spent in a body position; and expiry of the first part of the designated period.

45. A system for determining the occurrence of respiratory events, comprising:
a plurality of electrodes positionable at frontal locations on a person's forehead to sense bioelectric signals;
a measurement device for measuring respiratory flow of the person; and
a processing unit coupled to the plurality of electrodes and the flow measurement device, wherein the processing unit is configured to determine a sleep state of the person based on the bioelectric signals and to determine the occurrence of an arousal based on the bioelectric signals, and wherein the processing unit is further configured to determine the occurrence of an apnea event based on the sleep state and the respiratory flow and to determine the occurrence of a hypopnea event based on the respiratory flow, the sleep state of the person and the occurrence of an arousal.

46. A method of determining the occurrence of respiratory events, comprising:
positioning a plurality of electrodes at frontal locations on a person's forehead;
sensing bioelectric signals using the plurality of electrodes;
measuring respiratory flow of the person;
determining a sleep state of the person based on the bioelectric signals;
determining occurrence of an arousal based on the bioelectric signals;
determining occurrence of an apnea event based on the sleep state and the respiratory flow; and
determining occurrence of a hypopnea event based on the respiratory flow, the sleep state and the arousal.
